(19) United States
(12) Patent Application Publication
(10) Pub. No.: US 2017/0298441 A1
(43) Pub. Date: Oct. 19, 2017

(54) USE OF CLONAL EVOLUTION ANALYSIS FOR IBRUTINIB RESISTANCE IN CHRONIC LYMPHOCYTIC LEUKEMIA PATIENTS

(71) Applicants: DANA-FARBER CANCER INSTITUTE, INC, Boston, MA (US); BOARD OF REGENTS, THE UNIVERSITY OF TEXAS SYSTEM, Austin, TX (US); PRESIDENT and FELLOWS of HARVARD COLLEGE, Cambridge, MA (US)

(72) Inventors: Catherine J. WU, Brookline, MA (US); Dan-Avi LANDAU, New York, NY (US); Jan A. BURGER, Houston, TX (US); Ivana Bozic, Cambridge, MA (US); MARTIN NOWAK, Lincoln, MA (US)

(21) Appl. No.: 15/513,127

(22) PCT Filed: Sep. 22, 2015
(86) PCT No.: PCT/US2015/051340

§ 371 Date: Mar. 21, 2017

Related U.S. Application Data
(60) Provisional application No. 62/053,697, filed on Sep. 22, 2014.
(60) Provisional application No. 62/181,715, filed on Jun. 18, 2015.

(51) Int. Cl. C12Q 1/68; G01N 33/574

(52) U.S. Cl. CPC C12Q 1/6886; G01N 33/57426; G01N 33/574; C12Q 2600/156; C12Q 2600/106

(57) ABSTRACT

The present invention generally relates to the methods and use of clonal evolution analysis of the kinetics and genetic alterations associated with the development of resistance to a therapy using whole-exome and deep targeted sequencing in treating patients in need thereof.

### RELATED APPLICATIONS AND INCORPORATION BY REFERENCE

The present application is filed pursuant to 35 U.S.C. §371 as a U.S. National Phase Application of International Patent Application No. PCT/US15/51340, which was filed on Sep. 22, 2015. This application claims benefit of U.S. provisional patent applications Ser. No. 62/,053,697 filed Sep. 22, 2014 and Ser. No. 62/181,715, filed Jun. 18, 2015.

### STATEMENT AS TO FEDERALLY FUNDED RESEARCH

This invention was made with government support under Grant No. HG003067 awarded by the National Institutes of Health. The government has certain rights in the invention.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention generally relates to the methods and use of clonal evolution analysis of the kinetics and genetic alterations associated with the development of resistance to a therapy using whole-exome and deep targeted sequencing in patients in need thereof.

### BACKGROUND OF THE INVENTION

Many patients undergoing treatment or therapy for a disease develop resistance to the treatment. Such diseases share the characteristic of undergoing an evolutionary process to become resistant to the treatment or therapy. Therefore, there is a need to understand the mechanism of the evolutionary process in relation to resistance to therapy to prevent such resistance and future relapses.

Chronic lymphocytic Leukemia (CLL) is one example of disease where resistant clones lead to resistance to therapy and relapse. B cell receptor (BCR) signaling is a critical growth and survival pathway in several B cell malignancies, including CLL (1). BCR signaling can be abrogated by novel kinase inhibitors that target the BCR-associated kinases SYK (2), BTK (3), and PI3Kδ (4). The BTK inhibitor ibrutinib is a small molecule that inactivates BTK through irreversible covalent binding to Cys-481 within the ATP binding domain of BTK (5). In a recent trial in patients with relapsed/refractory CLL, ibrutinib induced an overall response rate of 71% and an estimated progression-free survival rate of 75% after 26 months of therapy (3). However, a small fraction of patients develop progressive CLL after initially responding to ibrutinib (3). Among these, patients carrying BTK mutations at the ibrutinib binding site (C481S) or affecting the BCR signaling-related molecule PLCγ2 (R665W, L845F, S707Y) were recently highlighted (6-8).

The ability of cancer cells to evolve and adapt to targeted therapies is a challenge that limits treatment success and durability of responses. Whole-exome sequencing (WES), along with analyses of clonal heterogeneity and clonal evolution in CLL, can provide insight into emergence and expansion of sub-clones that carry driver mutations (e.g., SF3B1 and TP53) under therapeutic pressure (9). However, these methods were unable to detect genetic heterogeneity within a cancer that are present at very low frequencies before treatment or therapy.

Therefore, it an object of the present invention to provide new methods for detecting clonal evolution and novel treatments based on clonal evolution.

Citation or identification of any document in this application is not an admission that such document is available as prior art to the present invention.

### SUMMARY OF THE INVENTION

It is an object of the present invention to provide a novel analytic framework, methods and systems that are widely applicable across disease, and specifically cancer. Clonal analysis to determine sub-populations of clones before treatment, as well as frequent serial clonal analysis can provide information regarding the clone specific decline/growth kinetics as they occur in patients. This type of analysis provides vital information regarding the fitness of different genetic lesions with and without therapy, which may be immensely beneficial to the design of the next generation of therapeutic approaches to overcome the evolutionary capacity of disease.

Applicants demonstrate the novel methods and systems of the present invention in CLL. To determine patterns of clonal evolution in ibrutinib-resistant CLL patients, Applicants performed a longitudinal genomic investigation of 5 CLL patients who achieved partial remissions and later experienced disease progression.

In one aspect, the present invention provides a method of individualized or personalized treatment for a disease undergoing clonal evolution and for preventing relapse after treatment in a patient in need thereof comprising: (a) determining mutations present in a disease cell fraction from the patient before administration of a therapy; (b) determining subclonal populations within the disease cell fraction; (c) selecting at least one subclonal population to treat; and (d) treating the patient with a therapy comprising administering at least one component, wherein each selected subclonal population does not contain a mutation associated with resistance to the at least one component of the therapy. In one embodiment, the method may further comprise determining mutations and subclonal populations on at least one time point after administration of the therapy.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIGS. 1A-1E illustrate evidence of clonal evolution with late disease progression following ibrutinib (Patient 1).

FIGS. 2A-2F illustrate clonal evolution with early disease progression following ibrutinib (Patients 2 and 3).

FIGS. 3A-3E illustrate Droplet-based detection of resistance subclones at the time of treatment initiation (Patients 1-3).

FIGS. 4A-4C illustrate histiocytic sarcoma transdifferentiation of CLL during ibrutinib therapy (Patient 5).

FIGS. 5A-5D illustrate the impact of del(8p) on apoptosis in response to ibrutinib and/or TRAIL in CLL.

FIG. 6 illustrates that two dimensional clustering enables the distinction of unique clones and the reconstruction of a phylogenetic tree.

FIG. 7 illustrates the complete mutation annotation for each patient overlaid on the phylogenetic tree.

FIG. 8 illustrates an IGV screenshot of the BTK mutation in Patient 4 CLL cells at the time of relapse.

FIG. 9 illustrates single cell droplet-PCR detection of resistance cells before and after ibrutinib exposure.

FIG. 10A-D. illustrates the characterization of del(8p) in ibrutinib-resistant CLL patients.

### DETAILED DESCRIPTION OF THE INVENTION

The following detailed description is of example embodiments of the presently claimed invention with references to the accompanying drawings. Such description is intended to be illustrative and not limiting with respect to the scope of the present invention. Such embodiments are described in sufficient detail to enable one of ordinary skill in the art to practice the subject invention.

The present invention provides a novel analytic framework, methods and systems that are widely applicable across diseases, and specifically different types of cancer. The present invention provides for the detection and grouping of subclonal populations of cells or disease causing entities based upon mutations present in each cell or disease causing entity. The subclones may be present in less than 10%, less than 5%, less than 1%, less than 0.1%, less than 0.01%, less than 0.001% or less than 0.0001% of the diseased cells or malignant cells. Not being bound by a theory, a novel treatment regimen can be formulated based on the presence of driver or resistance mutations present in each subclonal population. Not being bound by a theory, if two mutations are present in a population, but the mutations do not overlap in a subclonal population, a treatment or therapy targeting both unmutated alleles will be an effective treatment. Not being bound by a theory, if only one therapy is administered that targets only one of the unmutated alleles, then the subclonal population with a resistance mutation will not be eliminated. Not being bound by a theory, if a single subclonal population includes two mutations conferring resistance to two treatments, then treatment with drugs targeting both unmutated alleles would not be effective.

The present invention provides for detecting subclonal populations before treatment. The present invention also further provides for the detection of subclonal populations during and after the selected treatment. Not being bound by a theory, an initial therapy can be selected based upon the subclonal populations detected before treatment. After the initial treatment, clonal evolution in subclonal populations can be further monitored to adjust the treatment based on the clonal evolution determined. Clonal evolution can be determined at any time interval after initiation of treatment.

The disease can be any disease where drug resistance mutations occur or where clonal evolution occurs. The disease may be cancer. The cancer may include, without limitation, leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases. The disease may be a viral or bacterial infection. The viral infection may be HIV. Not being bound by a theory a single HIV virus particle infects a single cell and determining mutations of virus from single cells allows the detection of virus subclonal populations each containing different mutations.

In one aspect, the present invention provides a method of individualized or personalized treatment for a disease undergoing clonal evolution and for preventing relapse after treatment in a patient in need thereof comprising: determining mutations present in a disease cell fraction from the patient before administration of a therapy; determining subclonal populations within the disease cell fraction; selecting at least one subclonal population to treat; and treating the patient with a therapy comprising administering at least one component, wherein each selected subclonal population does not contain a mutation associated with resistance to the at least one component of the therapy. Mutations associated with resistance may be any mutation indicates that a subclonal population will become resistant to a therapy. The mutation may be in the target of the therapy or it may be in a gene that is determined to promote a mutation in the target of the therapy. Not being bound by a theory, the mutation may make it more likely that clonal evolution will produce resistance to a traditional therapy. Thus, the present invention provides novel therapies determined by the clonal evolution in a patient in need thereof.

The method may further comprise determining mutations and subclonal populations on at least one time point after administration of the therapy. The at least one time point may be a week, a month, a year, two years, three years, or five years after initiation of a therapy. The time point may be after a relapse in the disease is detected. Relapse may be any recurrence of symptoms of a disease after a period of improvement. Time points may be taken at any point after the initial treatment of the disease and includes time points following a change to the treatment or after the treatment has been completed.

The treatment may be adjusted if new mutations associated with resistance are detected in a subclonal population. In one embodiment, a therapy is chosen based on including components targeting subclonal populations that do not contain mutations associated with resistance to the therapy. After initiation of the treatment, clonal evolution analysis of the present invention may be performed at a time point. Minor subclonal populations containing mutations associated with resistance may become dominant. The treatment may then be adjusted based on this subclonal population. The subclonal populations selected for the initial therapy may have also obtained mutations associated with resistance to the therapy.

The selecting of at least one subclonal population to treat may comprise determining subclone-specific decline and/or growth kinetics, wherein the treatment is adjusted if there is an increase in at least one subclone. The therapy may comprise administering at least two components, wherein each selected subclonal population is targeted by at least one component of the therapy and wherein each selected subclonal population does not contain a mutation associated with resistance to at least one component of the therapy. The selecting at least one subclonal population to treat may comprise determining the copy number of each subclonal population. The mutations may be somatic mutations.

In another aspect, the present invention provides a method for treating or inhibiting a disease in a person in need thereof, comprising providing individualized or personalized treatment, comprising: (a) analyzing DNA from a blood, saliva or tissue sample obtained from the person; (b) analyzing clonal evolution in the sample; and (c) determining from said sample the presence somatic mutations in the clonal evolution. In one embodiment, the somatic mutation is present in a cancer. The somatic mutation can be any mutation associated with resistance to a treatment or therapy (See, e.g., www.mycancergenome.org). In one embodiment, the presence of a mutation in one or more genes selected from the group consisting of ATM, BRAF, DMBX1, del(8p), del(11q), del(13q), DNAJB14, EIF2A, EP300, MLL2, NRAS, RPS15, and SF3B1 indicates the person is Bruton's tyrosine kinase (BTK) inhibitor insensitive. Not being bound by a theory, the person who is BTK insensitive should be treated with at least one therapy in addition to or independent of a Bruton's tyrosine kinase (BTK) inhibitor.

In another embodiment of the invention, the method of determining subpopulations comprises (i) obtaining a blood, bone marrow or tissue sample from the person; (ii) isolating DNA from the blood, saliva or tissue sample; and (iii) genotyping the DNA. In a further embodiment, the tissue sample is a formalin-fixed, paraffin-embedded (FFPE) tissue section. In another further embodiment, the method wherein step (a) comprises whole-exome sequencing (WES) and/or genome-wide copy number profiling. In another embodiment, the method of any one of the preceding methods wherein step (c) comprises identifying somatic mutations with an algorithm (e.g, MuTech). In another embodiment, the method of any one of the preceding methods, wherein the step (c) comprises allele-specific analysis. In another embodiment, the method of any one of the preceding methods, wherein step (c) comprises deep sequencing and targeted re-sequencing with microfluidic PCR. In a further embodiment, the method wherein the allelic fractions are converted into cancer cell fractions (CCF). In another further embodiment, the method wherein the CCFs are clustered to delineate distinct subclonal populations that harbor multiple subclonal mutations and to infer the phylogenetic relationships between these populations.

In another embodiment, single cell analysis is used to determine gene mutations. Not being bound by a theory, single cell analysis allows the identification of single cells containing a mutation among a large population of cells. Not being bound by a theory, a mutation may be detectable by Deep sequencing if it is present in 2% of the cells in a population, whereas 1 cell in 500,000 may be detected using single cell analysis.

In one embodiment, single cell analysis is performed by digital polymerase chain reactions (PCR), e.g., Fluidigm C. Digital polymerase chain reaction (digital PCR, DigitalPCR, dPCR, or dePCR) is a refinement of conventional polymerase chain reaction methods that can be used to directly quantify and clonally amplify nucleic acids including DNA, cDNA or RNA. The key difference between dPCR and traditional PCR lies in that PCR carries out one reaction per single sample and dPCR carries out a single reaction within samples separated into a large number of partitions wherein the reactions are carried out in each partition individually. A sample is partitioned so that individual nucleic acid molecules within the sample are localized and concentrated within many separate regions. The capture or isolation of individual nucleic acid molecules may be effected in micro well plates, capillaries, the dispersed phase of an emulsion, and arrays of miniaturized chambers, as well as on nucleic acid binding surfaces.

In a preferred embodiment single cell sequencing is performed using microfluidics. Microfluidics involves micro-scale devices that handle small volumes of fluids. Because microfluidics may accurately and reproducibly control and dispense small fluid volumes, in particular volumes less than 1 μl, application of microfluidics provides significant cost-savings. The use of microfluidics technology reduces cycle times, shortens time-to-results, and increases throughput. Furthermore, incorporation of microfluidics technology enhances system integration and automation. Microfluidic reactions are generally conducted in microdroplets. The ability to conduct reactions in microdroplets depends on being able to merge different sample fluids and different microdroplets. See, e.g., US Patent Publication No. 20120219947.

Droplet microfluidics offers significant advantages for performing high-throughput screens and sensitive assays. Droplets allow sample volumes to be significantly reduced, leading to concomitant reductions in cost. Manipulation and measurement at kilohertz speeds enable up to 10⁸ samples to be screened in a single day. Compartmentalization in droplets increases assay sensitivity by increasing the effective concentration of rare species and decreasing the time required to reach detection thresholds. Droplet microfluidics combines these powerful features to enable currently inaccessible high-throughput screening applications, including single-cell and single-molecule assays. See, e.g., Guo et al., Lab Chip, 2012,12, 2146-2155.

The manipulation of fluids to form fluid streams of desired configuration, discontinuous fluid streams, droplets, particles, dispersions, etc., for purposes of fluid delivery, product manufacture, analysis, and the like, is a relatively well-studied art. Microfluidic systems have been described in a variety of contexts, typically in the context of miniaturized laboratory (e.g., clinical) analysis. Other uses have been described as well. For example, WO 2001/89788; WO 2006/040551; U.S. Patent Application Publication No. 2009/0005254; WO 2006/040554; U.S. Patent Application Publication No. 2007/0184489; WO 2004/002627; U.S. Pat. No. 7,708,949; WO 2008/063227; U.S. Patent Application Publication No. 2008/0003142; WO 2004/091763, U.S. Patent Application Publication No. 2006/0163385; WO 2005/021151 ; U.S. Patent Application Publication No. 2007/0003442; WO 2006/096571 ; U.S. Patent Application Publication No. 2009/0131543; WO 2007/089541; U.S. Patent Application Publication No. 2007/0195127; WO 2007/081385; U.S. Patent Application Publication No. 2010/0137163; WO 2007/133710; U.S. Patent Application Publication No. 2008/0014589; U.S. Patent Application Publication No. 2014/0256595; and WO 2011/079176. In a preferred embodiment single cell analysis is performed in droplets using methods according to WO 2014085802. Each of these patents and publications is herein incorporated by reference in their entireties for all purposes.

In another embodiment of the invention, the method of any one of the preceding methods wherein step (c) comprises immunohistochemical (IHC) staining, fluorescent in situ hybridization (FISH) chromosome analysis, and/or immunoglobulin hypervariable (IGHV) gene region mutation analysis. In a further embodiment, the method wherein the mutation in SF3B1 is pG742D. In another embodiment, the method wherein the mutation in TP53 is biallelic inactivation of TP53. In another embodiment, the method wherein the mutation in SF3B1 is p.K666T. In another embodiment, the method wherein the mutation in a PLCG2 mutation S707F, M1141R, M1141K and/or D993H. In another embodiment, the method wherein the mutation is a del(8p) mutation. In another embodiment, the method wherein the mutation is a driver mutations in EIF2A and/or RPS15. In another embodiment, the method wherein the mutation in EP300 is Y1397F. In another embodiment, the method wherein the mutation in MLL2 is Q3892. In another embodiment, the method wherein the mutation is in EJF2A and/or RPS15. In another embodiment, the method wherein the mutation is in EP300 and/or MLL2. In another embodiment, the method wherein the mutation is a del(11q) and/or del(13q) mutation. In a further embodiment, the method wherein the mutation is a ATM, BRAF and/or del[11q] mutation. In another embodiment, the method wherein the mutation in EP300 is N1511S.

In another aspect of the invention, the method of any one of the preceding methods wherein the therapy is chemotherapy, a monoclonal antibody, a targeted therapy, a stem cell transplant, leukapheresis, surgery, radiation therapy or a combination thereof. In a further embodiment, the method wherein the chemotherapy is a purine analog, an alkylating agent, a corticosteroid or other chemotherapy drug. In another embodiment, the method wherein the purine analog is bine (Fludara®), pentostatin (Nipent®), or cladribine (2-CdA, Leustatin®). In another embodiment, the method wherein the alkylating agent is chlorambucil (Leukeran®), cyclophosphamide (Cytoxan®), or bendamustine (Treanda®). In a further embodiment, the method wherein the corticosteroid is prednisone, methylprednisolone, or dexamethasone. In another embodiment, the method wherein the other chemotherapy drug is doxorubicin (Adriamycin®), methotrexate, oxaliplatin, vincristine (Oncovin®), etoposide (VP-16), or cytarabine (ara-C). In another embodiment, the method wherein the monoclonal antibody targets the CD20 antigen or the CD52 antigen. In another embodiment, the method wherein the monoclonal antibody is Rituximab (Rituxan), Obinutuzumab (Gazyva™), Ofatumumab (Arzerra®), or Alemtuzumab (Campath®). In a further embodiment, wherein the targeted therapy is Idelalisib (Zydelig®).

In an aspect of the invention, treatments directed towards CLL are described. One method of treatment is chemotherapy. Chemotherapy employs drugs to stop the growth of cancer cells by either killing the cells or inhibiting cells from dividing. Drugs approved for use for chemotherapy treatment in CLL include Alemtuzumab, Ambochlorin, (Chlorambucil). Amboclorin (Chlorambucil), Arzerra (Ofatumumab), Bendamustine Hydrochloride, Campath (Alemtuzumab), Chlorambucil, Ciafen (Cyclophosphamide), Cyclophosphamide, Cytoxan (Cyclophosphamide), Fludara (Fludarabine Phosphate), Fludarabine Phosphate, Gazyva (Obinutuzumab), Ibrutinib, Idelalisib, Imbruvica (Ibrutinib), Leukeran (Chlorambucil), Linfolizin (Chlorambucil), Mechlorethamine Hydrochloride, Mustargen (Mechlorethamine Hydrochloride), Neosar (Cyclophosphamide), Obinutuzumab, Ofatumumab, Treanda (Bendamustine Hydrochloride), and Zydelig (Idelalisib). Drug combinations used in CLL include chlorambucil-prednisone and cyclophosphamide-vincristine sulfate-prednisone (CVP).

Another treatment utilized in the treatment of CLL is targeted therapy. Targeted therapy is a type of treatment where the cancerous cells are specifically or preferentially attacked and normal/healthy cells are left unharmed. An example of targeted therapy is monoclonal antibody therapy which uses antibodies synthesized from a single type of immune system cell. The synthesized antibodies identify the cancerous cells or any substances which proliferate cancerous cell growth and attaches itself to the target. The antibodies either kill the cell or blocks it growth. Monoclonal antibodies are generally delivered by infusion and can be used alone or in combination with other methods of treatment.

Chemotherapy can also be used with stem cell transplant to treat CLL. This is a method of employing chemotherapy and replacing blood-forming cells destroyed by the cancer treatment. Once chemotherapy is completed in the patient, stem cells from either the patient (prior to chemotherapy) or a donor are reinfused into the patient and restore the body's blood cells.

Additionally, biological therapy (also sometimes referred to biotherapy or immunotherapy) can also be utilized in the treatment of CLL. Biological therapy uses the patient's own immune system to fight cancer. Naturally occurring substances within the body or synthesized substances are used to help the patient's body to fight cancer by boosting the immune system. In some types of therapy, the boosted immune system will attack or inhibit specific cancer cells, thereby inhibiting cancer proliferation.

In an aspect of the invention, treatments directed towards HIV are described. Treatments may be any antiretroviral therapy. These may be any combination of protease inhibitors, integrase inhibitors, and/or nucleoside analogues.

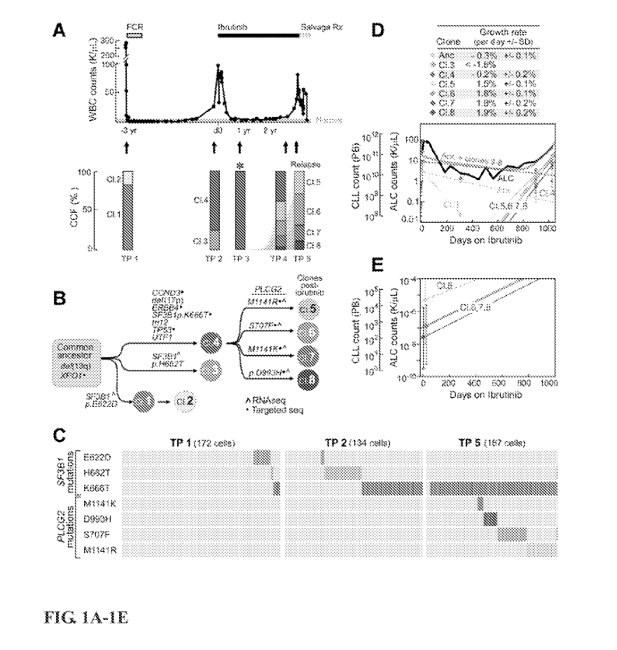

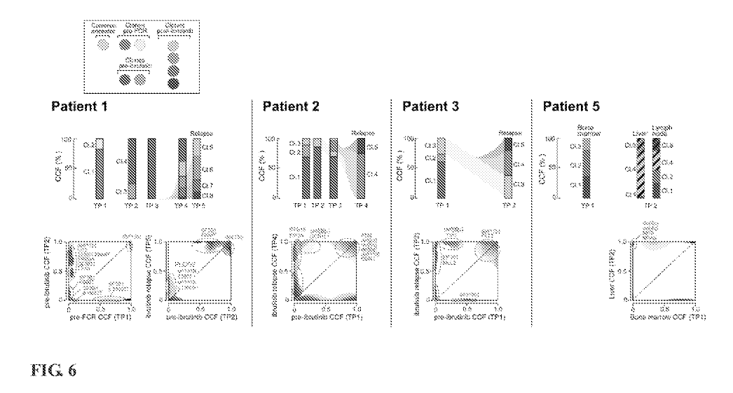

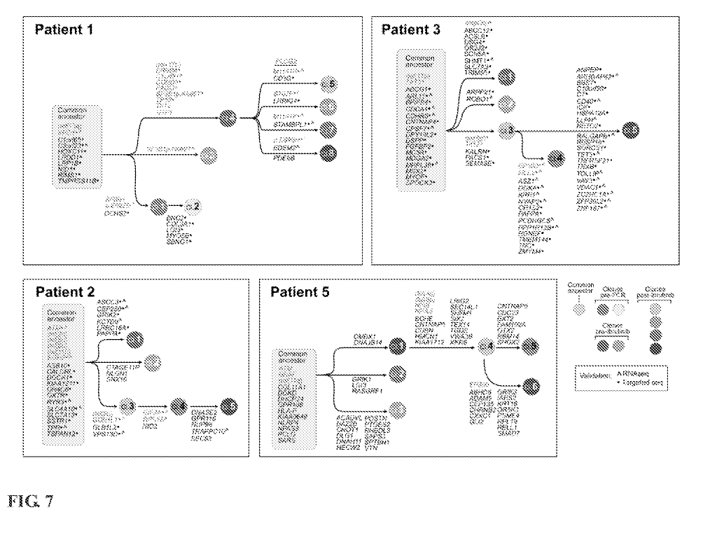

Turning to FIG. 1, evidence of clonal evolution with late disease progression before and following ibrutinib (Patient 1) is illustrated. In FIG. 1A, white blood cell counts and treatment course of Patient 1. Peripheral blood specimens were sampled at 5 time points (indicated by arrows), and CLL cells underwent whole exome sequencing. Following somatic mutation calling, cancer cell fraction (CCF) of somatic variants was inferred by ABSOLUTE analysis of deep sequencing data of the detected mutations (see Supplemental FIG. S1-S2). Asterisk-indicates that this sample had less purity, and hence clone sizes are estimates. In FIG. 1B, a phylogenetic tree was inferred based on PHYLOGIC, a novel algorithmic extension of ABSOLUTE. Driver mutations associated with each clone are indicated (a complete listing of somatic mutations and allelic fractions found for each clone in Supplementary Table S2 and FIG. 6-7). In FIG. 1C. multiplexed detection of somatic mutations in 134-172 single cells of Patient 1 at TP1, TP2 (pre-ibrutinib) and TP5 (ibrutinib relapse) are shown out of 192 assayed cells for each patient. Between all 3 time points, shifting cell subpopulations with SF3B1 mutation are observed. At TP5, SF3B1-K666T is detected in all cells, while the various PLCG2 mutations are detected in distinct subpopulations. FIG. 1D depicts the clonal kinetics during ibrutinib treatment. Filled circles—measurement of the number of cells comprising each subclone at each time point based on the subclone CCF and the corresponding absolute lymphocyte counts. Measurements are shown with 95% CI obtained from posterior distributions of CCFs. Empty circles—upper bound estimates (1% of total CLL cells) for subclones that were below the detection threshold of targeted deep sequencing. Solid lines denote predicted kinetics for clones detected on at least two measurements. Dashed lines represent kinetics with minimal absolute growth rates for clones detected in only one measurement. FIG. 1E. shows extrapolation of clone size with 95% CI at the time of treatment initiation for the PLCG2 mutated subclones.

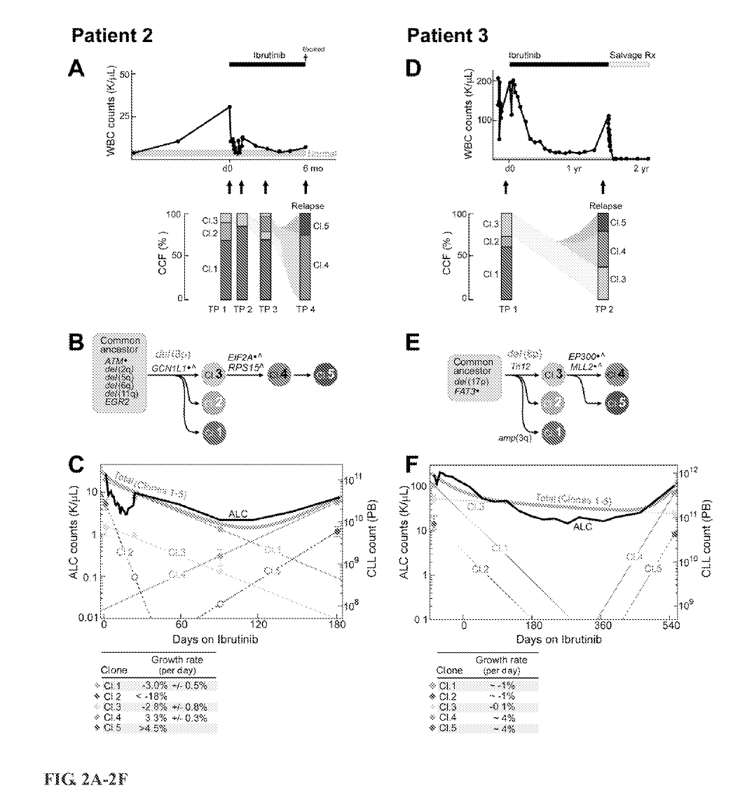

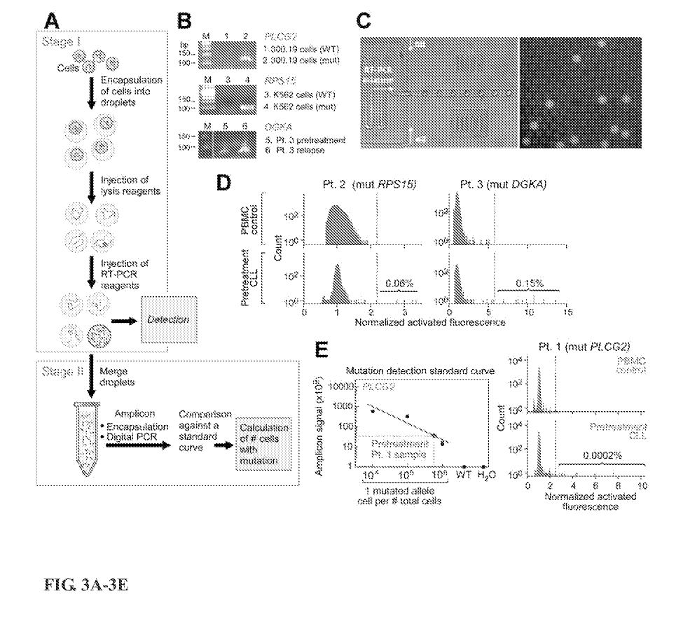

Turning to FIG. 2, which illustrates clonal evolution with early disease progression following ibrutinib in Patients 2 and 3, white blood cell counts and treatment courses of Patients 2 (FIG. 2A) and 3 (FIG. 2D) are shown. Peripheral blood specimens were sampled at serial timepoints (indicated by arrows), and CLL cells underwent whole-exome sequencing. Following somatic mutation calling, cancer cell fraction (CCF) of somatic variants were inferred by ABSOLUTE analysis (see FIGS. 6-7). The phylogenetic trees for Patient 2 (FIGS. 2B) and 3 (FIG. 2E) were inferred based on Phylogic. Driver mutations associated with each clone are indicated (a list of somatic mutations and allelic fractions found for each clone in FIGS. 6-7). Clonal kinetics during ibrutinib treatment for Patient 2 (FIG. 2C) and 3 (FIG. 2F). Filled circles—measurements combining clonal fractions and ALC counts. Empty circles are upper bound estimates (1% of total CLL cells) for clones that were below detection. Solid lines denote predicted kinetics for clones with at least two measurements. For Patient 2, dashed lines represent kinetics with minimal absolute growth rates for clones with only one measurement, while for Patient 3, the dashed lines represent kinetics obtained from fitting to absolute lymphocyte counts. Measurements are shown with 95% CI obtained from posterior distributions of CCFs. For Patient 3, Applicants assumed clones 1 and 2 have the same rates of decline and clones 4 and 5 have the same growth rates during treatment.

Turning to FIG. 3, droplet-based detection of resistance subclones at the time of treatment initiation (Patients 1-3) is shown. FIG. 3A. depicts a schema of the experimental workflow. FIG. 3B depicts the specificity of the mutation-detection primers visualized on an agarose gel in bulk cell line populations transfected to express minigenes encoding the wildtype (WT) vs mutated (MUT) allele (for PLCG2 and RPS15), or in bulk patient cDNA at pretreatment and relapse time points (Patient 3, DGKA) FIG. 3C. depicts a droplet apparatus, and detection of bright droplets following amplification. FIG. 3D depicts detection of mutated RPS15-specific single cells in Patient 2 samples and a PBMC control (left) and of mutated DGKA-specific single cells in Patient 3 samples and a PBMC control (right). FIG. 3E. depicts a standard curve for the detection of the PLCG2 M1141R template, established based on known input quantities on cell line (murine 30019 cells, with error bars shown) expressing the mutated template, and detection of PLCG2-M1141R in the pretreatment sample of Patient 1.

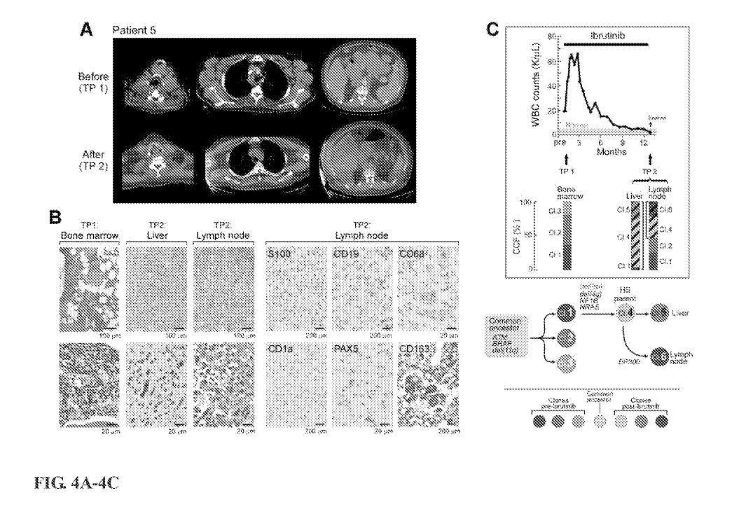

Turning to FIG. 4, histiocytic sarcoma transdifferentiation of CLL during ibrutinib therapy in Patient 5 is illustrated. In FIG. 4A, the regression of lymph node disease, visualized by CT scan, following ibrutinib exposure (at timepoint (TP) 2), compared to TP1. In FIG. 4B, TP2 (autopsy), histologic sections of liver and lymph node (LN), stained by H&E, showed histiocytic sarcoma with sheets of large atypical cells with irregular shaped nuclei, dense nuclear chromatin, and abundant cytoplasm (at ×100, and ×500 inserts). Occasional large neoplastic cells demonstrated 1 or 2 prominent eosinophilic nuclei. No lymphoid aggregates were seen. The neoplastic cells within the LN were strongly positive for CD163 and are negative for CD19, CD1a, and S100 protein (all at ×500). In FIG. 4C, white blood cell counts and clinical course for Patient 5. Whole-exome sequencing and CCF measurements were made prior to ibrutinib initiation (TP1) and from post-mortem specimens of the liver and lymph node (TP2). The fraction of cells that shared the mutations that define the histiocytic sarcoma parent clones are represented with black diagonal lines. Phylogenetic analysis was performed based on PHYLOGIC. A complete list of somatic mutations and allelic fractions for each clone is provided in FIGS. 6-7.

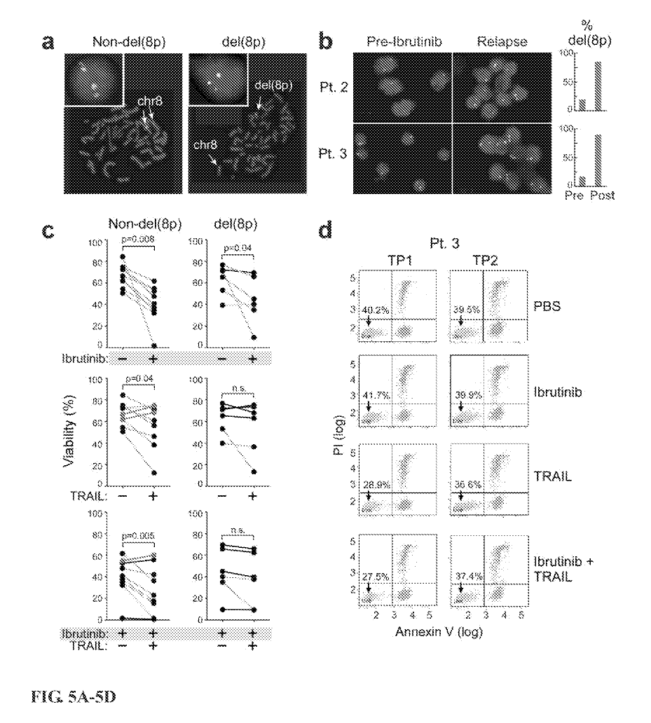

Turning to FIG. 5, the impact of del(8p) on apoptosis in response to ibrutinib and/or TRAIL in CLL is illustrated. FIG. 5A depicts representative interphase and metaphase FISH results following hybridization for probes specific for chromosome 8p21.3 (red) and chromosome 8 centromere (green), showing a CLL cell with a normal disomic hybridization pattern or with deletion of chromosome 8p. FIG. 5B depicts FISH hybridization of pretreatment and relapse samples from Patients 2 and 3 to detect del(8p). For each case, 100 nuclei were scored as summarized in the associated bar graphs. FIG. 5C depicts that primary CLL cells were isolated from peripheral blood and treated with ibrutinib and/or TRAIL at indicated concentrations. Cell death was assessed by Annexin V and Propidium Iodide (PI) staining and flow cytometry. p values calculated for absolute change in viability. In agreement with the known pleitropic effects of TRAIL on CLL cells (32), Applicants found that TRAIL treatment induced apoptosis in 7 of 9 of non-del(8p) samples, yet could also enhance survival in 2 of 9. Red—samples with a decrease in cell viability of at least 10% following exposure to TRAIL or ibrutinib. Purple—samples with increase in cell viability of at least 10% following exposure to TRAIL. Blue—Patient 3. FIG. 5D depicts cell viability measurements based on flow cytometric analysis following Annexin V and PI of CLL cells from Patient 3 before and after exposure to ibrutinib and/or TRAIL. Live cells constitute the double negative population.

Turning to FIG. 6, two dimensional clustering enables the distinction of unique clones and the reconstruction of a phylogenetic tree. The phylogenetic relationships were inferred using a serial implementation of 2 dimensional clustering (13) between every two samples in each patient. For each patient, the inferred patterns of clonal evolution are depicted (as in FIGS. 1-3), and representative examples of the 2 dimensional clustering are shown. The individual clones are highlighted with a circle, in addition to candidate driver mutations in each clone.

Turning to FIG. 7, complete mutation annotation overlaid on the phylogenetic tree is illustrated for Patients 1-3 and 5. For each patient, the mutations are assigned to each clone based on the phylogenetic inference resulting from the serial implementation of 2 dimensional clustering between every 2 samples. Likely candidate drivers are highlighted in pink.

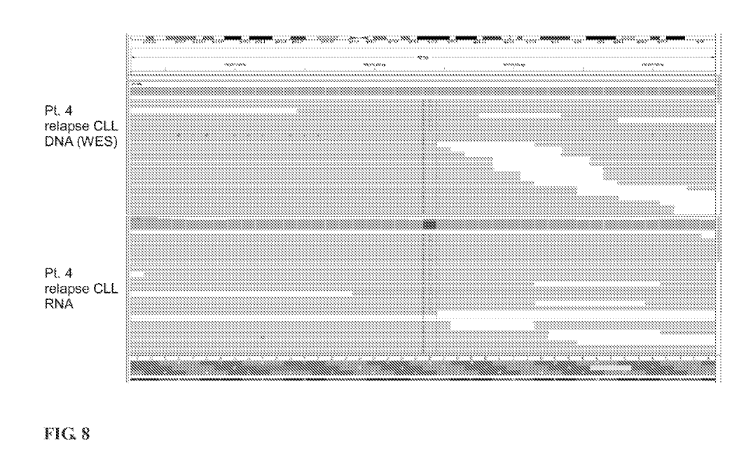

Turning to FIG. 8, illustrated is an IGV screenshot of the BTK mutation in Patient 4 CLL cells at the time of relapse. The BTK C481S mutation can be readily detected by both WES of relapsed leukemia cells (top), as well as by matched RNA-sequencing (bottom). Sequence is shown in reverse orientation. These data show a mutation that converts a cysteine at position 481 (TGC) to a serene (TCC).

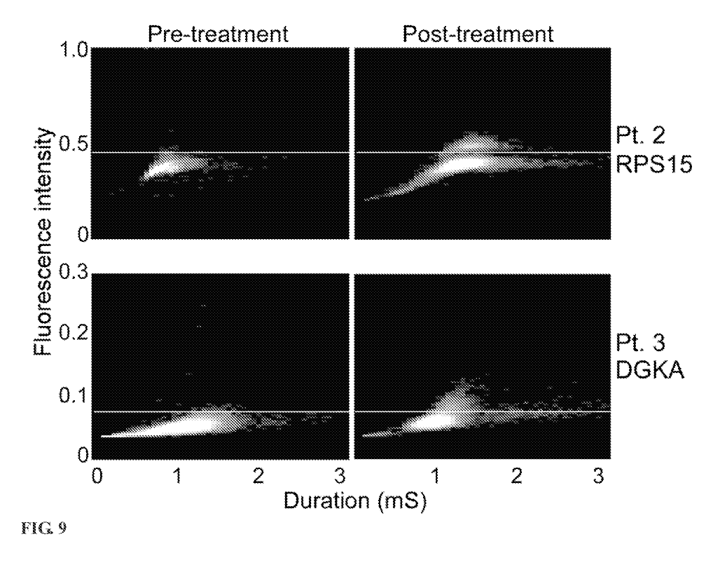

Turning to FIG. 9, single cell droplet-PCR detection of resistance cells before and after ibrutinib exposure is shown.

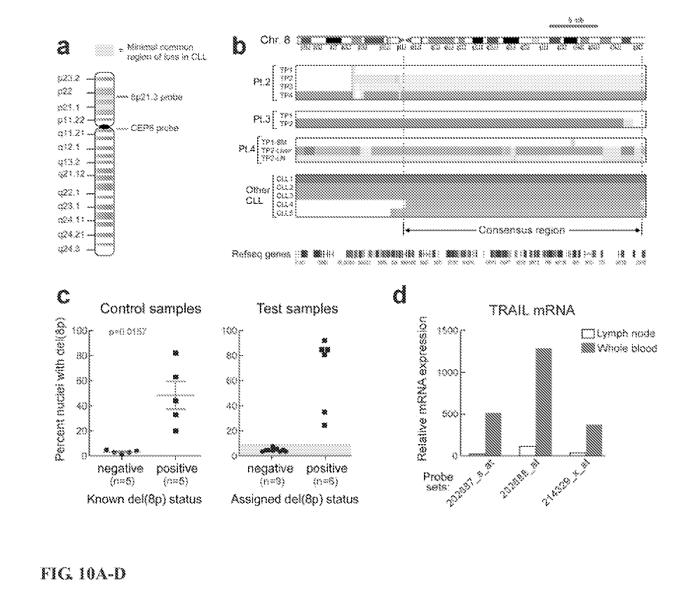

Turning to FIG. 10, illustrated is the characterization of del(8p) in ibrutinib-resistant CLL patients. FIG. 10 A. depicts a schematic of the minimal common region of loss of chromosome 8p in CLLs, to which FISH probes were designed. FIG. 10B. depicts SNP array analysis of Patients 2, 3 and 5 and other CLLs from DFCI (CLL1-CLL5) to which deletion in chromosome 8p was detected. FIG. 10C depicts the percentage of nuclei scored with 8p deletion following hybridization to the 8p FISH probe across nuclei from CLL samples, previously characterized by karyotyping as monosomy 8 or deletion of 8p by the BWH Clinical Cytogenetics lab as positive (n=5) or negative (n=5) for deletion in chromosome 8p. For each case, 100 nuclei were scored. The maximum background (i.e. a single 8p21.3 signal) in negative control specimens was 5%. Based on this data, the threshold for considering a sample as positive for del(8p) by FISH is 9.4% (mean of negatives+3SD). FIG. 10D. depicts the confirmation of del(8p) status of 9 ‘negative’ and 6 ‘positive’ samples (corresponding to the samples analyzed in FIG. 5C) through del(8p) FISH of fixed cell pellets.

The present invention provides many advantages. Analyses of subclonal populations before treatment and further analysis of serial samples from CLL patients developing resistance to the BTK inhibitor ibrutinib reveal a selection and expansion of pre-treatment resistant sub-clones carrying del(8p) and additional driver mutations, already present at the initiation of ibrutinib therapy. These findings of clonal evolution following therapy provide a novel mechanism for ibrutinib resistance, which previously has been attributed solely to mutations in BTK and related pathway molecules. Applicants novel finding that the mutations are already present at the initiation of therapy provides a paradigm shift that provides novel treatment regimens for treating any disease where resistance mutations are found. Further, these mutations indicate that based on clonal evolution subclonal populations may lead to drug resistance and effect patient outcome.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Methods

Patients were treated at MD Anderson Cancer Center (MDACC) on clinical trials approved by and conducted in accordance with the Institutional Review Board (IRB) of the University of Texas MDACC guidelines and with the principles of the Declaration of Helsinki. DNA was extracted from CD19⁺ enriched lymphocytes from bone marrow or blood, or from formalin-fixed, paraffin-embedded (FFPE) tissue sections (Patient 5). Matched germline DNA was isolated from matched granulocytes or unaffected FFPE tissue. Immunohistochemical (IHC) stains, fluorescent in situ hybridization (FISH) chromosome analyses, and immunoglobulin hypervariable (IGHV) gene region mutation analyses were done using routine procedures at MDACC. DNA samples were subject to whole-exome sequencing (WES) on Illumina GA-II sequencers (138X average sequencing depth (sequencing depth: average (mean) vs. median+IQR)) and genome-wide copy number profiling with the Human SNP Array 6.0 (Affymetrix), according to the manufacturer's protocol (Genetic Analysis Platform, Broad Institute, Cambridge Mass.).

The MuTect algorithm (www.broadinstitute.org/cancer/cga/mutect) was used to identify somatic mutations in targeted exons (10). All somatic mutations were reviewed manually from their respective BAM files using the Integrative Genomics Viewer (11). Somatic copy number alterations (SCNAs) were inferred from the whole-exome data from the ratio of tumor read depth to the expected read depth derived from a panel of normal samples using the CapSeg program and subsequently combined with allelic ratios to identify copy neutral loss using Allelic CapSeg (see Supplemental Information). In addition, for Patients 1 and 3, Human SNP Array 6.0 (Affymetrix) data with allele-specific analysis were also performed and allowed for the identification of copy-neutral LOH events and quantification of the homologous copy-ratios (HSCSs) [HAPSEG](12). Copy number changes were highly consistent between the two methods. Regions with germline copy number variants were excluded from the analysis. When DNA was available (Patients 1-4), we performed deep sequencing (median of 1997X and an interquartile range of 781X-2768X) in tumor and matched normal samples of detected mutations by targeted re-sequencing using microfluidic PCR (Access Array System, Fluidigm) together with Illumina Miseq. ABSOLUTE pipeline was implemented as previously described (9, 13) to the sequencing data to convert allelic fractions to cancer cell fractions (CCF) accounting for sample purity and local copy number information. The CCFs were clustered as previously described (9) to delineate distinct subclonal populations that harbor multiple subclonal mutations and to infer the phylogenetic relationships between these populations. The CCF of each clone was converted to clonal size (in cell number), by multiplying the CCF by the total size of the circulating CLL population as measured as the absolute lymphocyte counts/μl×the total blood volume. Clone-specific growth/decline rates were then interred by regression analysis applied to the measurements available for each subclone, assuming fixed exponential growth rates. Detection and quantification of single ibrutinib-resistant CLL cells was carried out using a droplet microfluidic approach in which targeted mutation-specific RT-PCR was performed. In vitro testing of cell viability of CLL cells with or without del(8p) (FISH confirmed using a probe targeting the minimal common region of deletion) following exposure to ibrutinib and/or TRAIL was performed by flow cytometry using Annexin-V and propidium iodide staining. Further information regarding WES and RNA-sequencing methods and additional methodological and analytical details are provided in Supplementary Information.

### CLL Samples from Patients Treated with Ibrutinib

All five patients were treated at MD Anderson Cancer Center (MDACC) and were enrolled on clinical trials approved by and conducted in accordance with the Institutional Review Board (IRB) of the University of Texas MDACC guidelines and with the principles of the Declaration of Helsinki. Patients 1, 3, 4 and 5 were treated on a phase Ib/II multicenter study of ibrutinib (NCT01105247), while Patient 2 was enrolled on a single-center phase II clinical trial of ibrutinib and rituximab (NCT01520519). Heparinized blood was collected before and after initiation of ibrutinib therapy, and peripheral blood mononuclear cells (PBMCs) from patient samples were isolated by Ficoll/Hypaque density-gradient centrifugation, cryopreserved with 10% DMSO, and stored in vapor-phase liquid nitrogen until the time of analysis. For Patients 1-4, non-lymphoid cells (neutrophils) were isolated by subjecting the non-PBMC cells (following Ficoll separation) to hypotonic erythrocyte lysis (33). For Patient 5, DNA was extracted from 30 micron sections collected from patient marrow biopsy specimens, obtained as part of routine clinical care, or from formalin-fixed, paraffin-embedded (FFPE) liver, heart, and lymph node at the time of autopsy.

### Immunohistochemical Analysis, Prognostic Markers, IGHV Analysis

Immunohistochemical (IHC) stains were performed on FFPE sections of tissue, or bone marrow core biopsies or clots using the avidin-biotin-peroxidase complex method and an automated immunostainer (Ventana-Biotech, Tucson, Ariz.). All tissue sections underwent heat-induced antigen retrieval before staining with antibodies. The pretreatment evaluation of all patients included fluorescent in situ hybridization (FISH) for common CLL chromosome abnormalities by the MDACC clinical laboratory, using a Vysis multicolor probe panel (Abbott Laboratories, Abbott Park, Ill.) designed to provide simultaneous detection of the 11q22.3 (ATM gene) region of chromosome 11; the 17p13.1 (TP53 gene) region of chromosome 17; the alpha satellite, centromeric region of chromosome 12 (D12Z3); the D13S319 locus (located between RBI and D13S25 loci) in the 13q14.3 region of chromosome 13; and the 13q34 region (LAMP1 gene) near the subtelomere of chromosome 13q in two hybridizations (two and three probes per hybridization, respectively), as previously described (34). A total of 200 interphase cells were analyzed for each probe. Positive patient cases were those with 5% or more of cells with the abnormality. Patients' FISH results were categorized according to the Darner hierarchy (35). Analysis of the mutation status of the IgV_{H} mutation status (MS) was done in the MDACC clinical laboratory, according to established protocols described before (36).

To detect deletions of chromosome 8p, interphase FISH was performed on fixed cell pellets stored at −20° C., obtained from conventional cytogenetic analysis, or cytospins. The cytospins were generated with 5×10⁴ CLL cells (Shandon cytospins; 700 rpm for 5 minutes) fixed with methanol:acetone (3:1) at room temperature for 10 minutes and then washed with 70% ethanol. Hybridization, using a probe cocktail consisting of Vysis LSI LPL probes targeting 8p21.3 (Abbott Molecular, Des Plaines, Ill.) and Vysis CEP8 (D8Z2) (Abbott Molecular, Des Plaines, Ill.), was performed according to the manufacturer's specifications. One hundred nuclei were scored per slide. Cut-offs for detection of 8p deletion or monosomy 8 were calculated using negative controls specimens with matching karyotype information, based on 3-standard deviations from the mean. The specific cut-off for 8p21.3 deletion was 9.4%.

Immunohistochemical (IHC) stains were performed on FFPE sections of tissue, or bone marrow core biopsies or clots of Patient 5 using the avidin-biotin-peroxidase complex method and an automated immunostainer (Ventana-Biotech, Tucson, Ariz.). All tissue sections underwent heat-induced antigen retrieval before staining with antibodies. Sequence analysis of the immunoglobulin hypervariable (IGHV) gene region in samples with histologic evidence of histiocytic sarcoma (Patient 5) was performed on DNA extracted from FFPE tissue sections. To determine the degree of somatic mutation in the IGHV region of non-hematopoietic tissues, patient's IGHV sequences were aligned to germline sequences and the patient's known previously characterized IGHV sequence (VH3-09), using the international ImMunoGeneTics (IMGT) information system and database tools (IMGT/V-Quest, imgt.org). As per convention, the IGHV somatic mutation status was designated as unmutated if there was ≧98% homology; or as mutated if there was <98% homology to germline sequences (37).

### Nucleic Acid Extraction and Quality Control

For Patients 1-3 and 5, genomic DNA was extracted from CLL PBMC and matched neutrophils (Qiagen). DNA analyses were done after informed consent under IRB-approved research protocols between MDACC and the Broad Institute Tumor and normal DNA concentration were measured using PicoGreen dsDNA Quantitation Reagent (Invitrogen, Carlsbad, Calif.). For Patient 4 samples, paraffin was removed from samples using Citrisolv and several ethanol washes, and then cells were lysed overnight at 56° C. DNA. After removal of DNA crosslinks through incubation at 90° C., DNA extraction was performed (QIAamp DNA FFPE Tissue Kit, Qiagen). A minimum DNA concentration of 60 ng/ml was required for sequencing. All Illumina sequencing libraries were created with the native DNA. The identities of all tumor and normal DNA samples were confirmed by mass spectrometric fingerprint genotyping of 24 common SNPs (Sequenom, San Diego, Calif.). RNA from CLL-B cells was extracted using standard protocols (RNAeasy kit, Qiagen).

### Whole-Exome Sequencing

Library construction from CLL and matched germline DNA of Patients 1-5 was performed as described in Fisher et al. (38), with the following modifications: (i) initial genomic DNA input into shearing was reduced from 3 μg to 10-100 ng in 50 μL of solution; (ii) For adapter ligation, Illumina paired end adapters were replaced with palindromic forked adapters (from integrated DNA Technologies), with unique 8 base molecular barcode sequences included in the adapter sequence to facilitate downstream pooling. With the exception of the palindromic forked adapters, the reagents used for end repair, A-base addition, adapter ligation, and library enrichment PCR were purchased from KAPA Biosciences in 96-reaction kits. (iii) During the post-enrichment SPRI cleanup, elution volumes were reduced to 20 μL to maximize library concentration, and a vortexing step was added to maximize the amount of template eluted. Any libraries with concentrations below 40 ng/μl (per PicoGreen assay, automated on an Agilent Bravo) were considered failures and reworked from the start of the protocol. Following library construction, hybridization and capture were performed using the relevant components of Illumina's Rapid Capture Exome Kit and following the manufacturer's suggested protocol. All hybridization and capture steps were automated on the Agilent Bravo liquid handling system. Based on qPCR quantification with probes specific to the ends of the adapters (KAPA Biosystems), libraries were normalized to 2 nM, then denatured using 0.1 N NaOH on the Perkin-Elmer MiniJanus. After denaturation, libraries were diluted to 20 pM (hybridization buffer, Illumina).

Cluster amplification of denatured templates was performed according to the manufacturer's protocol (Illumina) using HiSeq v3 cluster chemistry and HiSeq 2500 flowcells. Flowcells were sequenced on HiSeq 2500 using v3 Sequencing-by-Synthesis chemistry, then analyzed using RTA v.1.12.4.2 or later. Each pool of whole exome libraries was run on paired 76 bp runs, with and 8 base index sequencing read was performed to read molecular indices, across the number of lanes needed to meet coverage for all libraries in the pool. Alignments to hg19 using bwa version 0.5.9-r16 (39) and quality control were performed using the Picard (picard.sourceforge.net/) and Firehose (dx.doi.org/10.7908/C180514N) pipelines at the Broad Institute. Firehose is a framework combining workflows for the analysis of cancer sequencing data. The workflows perform quality control, local realignment, mutation calling, small insertion and deletion identification, rearrangement detection, and coverage calculations, among other analyses.

### Mutation Calling

The MuTect algorithm (www.broadinstitute.org/cancer/cga/mutect) was used to identify somatic mutations in targeted exons data (40). MuTect identifies candidate somatic mutations by Bayesian statistical analysis of bases and their qualities in the tumor and normal BAM files at a given genomic locus. The lowest allelic fraction at which somatic mutations could be detected on a per-sample basis was estimated based on cross-contamination level of 2%. All somatic mutations were reviewed manually using the Integrative Genomics Viewer (41).

### Somatic Copy Number Alteration Identification

Somatic copy number alterations (SCNAs) were identified from the analysis of genome-wide copy number profiles of CLL and matched germline DNA, obtained using the Genomewide Human SNP Array 6.0 (Affymetrix), according to the manufacturer's protocol (Genetic Analysis Platform, Broad Institute, Cambridge Mass.). SNP array data were deposited in dbGaP (phs000435.v1.p1). Alternatively SCNAs were inferred from the whole exome data from the ratio of tumor read depth to the expected read depth derived from a panel of normal samples using the CapSeg program (A. McKenna., B. Hernandez, M. Meyerson, G. G., and S. L. C., unpublished data). Allele-specific analysis allowed for the identification of copy neutral events and quantification of the homologous copy-ratios (HSCSs) using both Hapseg (42) on SNP arrays and Allelic CapSeg on exomes. Regions with germline copy number variants were excluded from the analysis.

### Deep Sequencing of Somatic Single Nucleotide Variants

When DNA was available, deep sequencing was performed by targeted resequencing using microfluidic PCR (Access Array System, Fluidigm). In total, 112/133 candidate somatic mutations identified in Patients 1-4 were sequenced with this approach. Tumor and matched normal samples were included in this analysis to exclude germline variants. Target-specific primers were designed to flank sites of interest and produce amplicons of 200 by ±20 bp. Per well, molecularly barcoded, Illumina-compatible specific oligonucleotides containing sequences complementary to the primer tails were added to the Fluidigm Access Array chip together with genomic DNA samples (20-50 ng of input) such that all amplicons for a given DNA sample shared the same index, and PCR was performed according to the manufacturer's instructions. From each individual collection well from the Fluidigm chip, indexed libraries were recovered for each sample, quantified using picogreen, and then normalized for uniformity across libraries. Resulting normalized libraries were loaded on the MiSeq instrument and sequenced using paired-end 150 bp sequencing reads (43). Mean coverage per sample is listed in supplemental Table 1 (range 938.9-5419.5X).

### ABSOLUTE Analysis and Deductive Logic of Clonal Evolution Mapping

ABSOLUTE pipeline was implemented as previously described (44, 45) to the sequencing data to convert allelic fractions to cancer cell fractions (CCF) accounting for sample purity and the local copy number information. The CCF's were clustered as previously described (45) to delineate distinct subclonal populations. Phylogenetic relationships between these populations were inferred using patterns of shared mutations and CCF, as previously described (46). The CCF of each clone was converted to clonal size (in cell number), by multiplying the CCF by the total size of the circulating CLL population (as measured by the absolute lymphocyte counts per microliter times the total blood volume). Clone-specific growth/decline rates were then inferred by using regression applied to the measurements available for each subclone, assuming fixed exponential growth rates.

### Mathematical Analysis of Clonal Kinetics

CCFs obtained from the ABSOLUTE analysis were combined with ALC counts to obtain estimates for the numbers of cancer cells in each clone present at the time of sequencing, assuming 51 as the peripheral blood volume (47). Applicants assumed that during treatment clones either grow or decline exponentially, with constant rates. For clones with exactly two measurements, standard deviations of growth rates were estimated using posterior distributions of CCFs. For clones with more than two measurements, we report standard errors for growth rates Obtained from linear regression in the log domain. We estimated the number of cells in a resistant clone at the time of initiation of ibrutinib treatment under the assumption that the growth rate of the resistant clone remains constant during treatment. Confidence intervals are obtained using posterior distributions of CCFs.

### Droplet-Based Detection of Single Cells with Somatic Gene Mutations by Real-Time RT-PCR

CLL cells, PBMC or cell lines resuspended in RPMI 1640 with 20% FBS were applied to polydimethylsiloxane (PDMS) microfluidic devices that were fabricated using standard soft lithographic methods (48). These microfluidic chips contain a co-flow droplet generator (cross-section of 35 μm²⁾ to yield 50 μm monodisperse aqueous drops in fluorinated oil, HFE-7500 (3M, St Paul, Minn.) containing 2% (w/w) Krytox-PEG diblock co-polymer surfactant (RAN Biotech, Beverly, Mass.). The microfluidic channel walls were rendered hydrophobic by treating them with Aquapel (PPG, Pittsburgh, Pa). 2× cell lysis buffer (1M Tris-HCl pH 8.0, 10% Tween-20 and 100 mg/ml proteinase K in one channel and a suspension of a single cell population or mixtures of cell populations are encapsulated together in drops via co-flow at a 1:1 ratio. The droplets were collected in 200 μl in a PCR tube and covered with mineral oil. Cell lysis within the drops was achieved using the following conditions: 37° C. for 10 min, 50° C. for 20 min, 70° C. for 10 min. Subsequently, the droplets containing single lysed cells were maintained on ice.

To amplify transcripts with the mutated alleles, the droplet suspension (at 33 pL volume per droplet) was introduced into a microfluidic pico-injection device and injected droplet by droplet with a 50 μL of a 2×RT-PCR cocktail through electro-coalescence (49). The 2×RT-PCR cocktail contained 4 μL of OneStep RT-PCR enzyme mix with 2×OneStep RT-PCR buffer (Qiagen, Valencia, Calif.) 800 μM dNTPs, 0.6 μM forward and reverse primers for patient-specific somatic mutations (purchased from IDT, Coralville, Iowa), 0.5 μM Taqman probe (Life Tech, Grand Island, N.Y.), 0.4 μg/μL BSA and 0.4% Tween 20.

Droplets were spaced on the chip by oil with 2% w/w surfactant. The device electrodes were connected to a high voltage TREK 2210 amplifier (TREK, Lockport, N.Y.) which supplies a 100 V sine wave at a frequency of 25 kHz. The flow rate of the PCR cocktail was chosen to ensure that the buffer would be added at ˜1:1 ratio upon coalescence. Typical flow rates fulfilling these requirements were 300 μL/hr for oil with surfactant, 60 μL/hr for the droplets containing lysed cells and 30 μL/hr for the PCR cocktail. The droplets were collected in a PCR tube and covered with mineral oil to prevent evaporation. RT-PCR was performed using the following conditions: 50° C. for 30 min, 95° C. for 10 min, 2 cycles of 94° C. for 15 s and 64° C. for 8 min, and 38 cycles of 95° C. for 15 s and 62° C. for 1 min.

Amplified mutated transcripts within single cells were detected by microfluidic-based sorting and signal detection. We re-injected the post-amplification drops, achieving a stream of evenly spaced drops through co-flowing of the drop suspension (flow rate 15 μL/h) and HFE-7500 oil with 1% surfactant (flow rate 180 μL/h) into a “T” junction. This stream flowed through a 25 μm×25 μm channel, and was exposed to an excitation laser (488 nm). Fluorescence information from single cells was collected by a microscope objective and focused onto a photomultiplier tube (PMT) (Hammamatsu). The pulses were acquired by a real-time field-programmable gate array card (National Instruments, Austin, Tex.), recorded by a LabView program and analyzed in MATLAB. The pulse height was used as the measure of droplet fluorescence. The pulse width, which is the duration of time for a drop to pass through the laser was used as the measure of droplet size. The sensitivity of our PMT was sufficiently high to detect droplets not containing target templates, due to the intrinsic fluorescence of the Tallman probe. Cells were designated as positive of the normalized activated fluorescence was higher than the signal generated by control PBMC for healthy adult volunteers.

For detection of very rare cells, Applicants developed a second step of droplet analysis using digital PCR. To obtain the templates for the second-round digital PCR, 25 μL of 1H,1H,2H,2H-perfluoro-1-octanol (PFO; Sigma-Aldrich, St. Louis, Mo.) was added to the pool of emulsion droplets and gently centrifuge to separate the phases, such that the PCR products from the first-round RT-PCR were in the liquid phase. PCR products were then diluted 1,000-fold, and 1 μL of the resulting product was encapsulated at a single template per droplet using a microfluidic device that contains a flow-focusing droplet maker with a cross-section of 15 μm×25 μm to generate 25 μm monodisperse aqueous drops in HET-7500 containing 2% (w/w) surfactant. The flow is driven by applying a −0.4 PSI vacuum at the outlet. The templates were then amplified using a 25 μL PCR cocktail containing 1 μL of OneStep RT-PCR, enzyme mix with 1×OneStep RT-PCR buffer (Qiagen), 400 μM dNTPs, 0.25 μM forward and reverse primers, 0.24 μM Taqman probe, 0.2 82 g/μL BSA, and 0.2% Tween-20 using the following RT-PCR protocol: 95° C. for 10 min, 40 cycles of 2 cycles of 94° C. for 15 s, 64° C. for 8 min, and 38 cycles of 95° C. for 15 s, 62° C. for 1 min. To quantify the mutant cells in the original sample, we compared fluorescence obtained from the experimental sample against a standard curve generated by the fluorescence detection from known mixtures of specific cell lines generated to express the gene of interest with or without the mutation of interest (FIG. 3E).

### RNA-Sequencing (RNA-seq)

5 μg of total RNA was poly-A selected using oligo-dT beads to extract the desired, mRNA, treated with DNase, and then processed with SPRI (Solid Phase Reversible Immobilization) beads according to the manufacturer's protocol. The selected Poly-A RNA was then fragmented into 450 bp fragments in an acetate buffer at high heat. Fragmented RNA was cleaned with SPRI and primed with random hexamers before first strand cDNA synthesis. The first strand was reverse transcribed off the RNA template in the presence of Actinomycin D to prevent hair-pinning followed by SPRI bead purification. The RNA in the RNA-DNA complex was then digested using RNase H. The second strand was next synthesized with a dNTP mixture in which dTTPs had been replaced with dUTPs. After another SPRI bead purification, the resultant cDNA was processed using Illumina library construction according to manufacturer's protocol (end repair, phosphorylation, adenylation, and adaptor ligation with indexed adaptors) SPRI-based size selection was performed to remove adaptor dimers present in the newly constructed cDNA library. Libraries were treated with Uracil-Specific Excision Reagent (USER) to nick the second strand at every incorporated Uracil (dUTP). Subsequently, libraries were enriched with 8 cycles of PCR using the entire volume of sample as template. After enrichment, the library is quantified using pico green, and the fragment size is measured using the Agilent Bioanalyzer according to manufactures protocol. Samples were pooled and sequenced using either 76 or 101 bp paired end reads.

RNaseq BAMs were aligned to the hg19 genome using the TopHat suite. Each somatic base substitution detected by WES was compared to reads at the same location in RNaseq. Based on the number of alternate and reference reads, a power calculation was obtained with beta-binomial distribution (power threshold used was greater than 80%). Mutation calls were deemed validated if 2 or greater alternate allele reads were observed in RNA-Seq at the site, as long as RNaseq was powered to detect an event at the specified location (Power >0.8).

### Cloning of Minigenes of PLCG2 and RPS15 and Generation of Mutation-Expressing Cell Lines

To generate stable cell lines expressing wild-type and mutant PLCG2 and RPS15, cDNA fragments around the mutation sites of interest were cloned. Mutations were introduced into the cDNA fragments through site-directed mutagenesis (Quickchange II Site-Directed Mutagenesis Kit, 200523-5, Agilent Technology). The vectors were linearized by MfeI, and transfected into murine 300.19 cells through electroporation. The transfected cells were selected with antibiotics for 2 weeks to generate the stable cell lines.

### Single Cell Detection of Patient Tumor-Specific Mutations

FACS-sorted CD19⁺CD5⁺7AAD⁻ single cells were collected and processed through the preamplification step as described by Livak el al. (50) with the exception that Reverse Transcription Master Mix (Fluidigm 100-6297) was used in the reverse transcriptase step and 5× PreAmp Master Mix (Fluidigm 100-5744) was used in the preamplification step. The use of a 5× formulation enabled reducing the volume of the preamplification reaction to 10 μL, which enhanced sensitivity. Paired mutated- and normal-allele specific primers were designed using a nested design with outer primers for preamplification and inner primers for qPCR detection, such that amplification of the mutated alleles with the two assays yielded a difference of at least 6 cycles. Each assay consisted of an allele-specific SuperSelective primer (51) and a common primer shared by the normal and mutation assay. The sequences of the primers used are provided in Supplemental Table S5. Single cell cDNA was submitted for multiplexed preamplification with a mixture of all the outer primers for patient-specific, mutation-specific assays at a final concentration of 50 nM each primer. Preamplified cDNA samples from single cells were then analyzed by qPCR using 96.96 Dynamic Array™ IFCs and the Biomark™ HD System from Fluidigm, per the manufacturer's procedures. For detection of somatic mutations, a Master Mix was prepared consisting of 420 μL 2× Fast-Plus EvaGreen Master Mix with Low ROX (Biotium 31014), 42 μL 20×DNA Binding Dye Sample Loading Reagent, 1.5 μL 500 mM EDTA, and 16.5 μL H₂O, and 4 μL of this mix was dispensed to each well of a 96-well assay plate. Three microliters of preamplified cDNA sample was added to each well and the plate was briefly vortexed and centrifuged. Following priming of the IFC in the IFC Controller HX, 5 μL of the cDNA sample+Master Mix were dispensed to each Sample Inlet of the 96.96 IFC. After loading the assays and samples into the in the IFC Controller HX, the IFC was transferred to the Biomark HD and PCR was performed using the thermal protocol GE Fast 96×96 PCR+Melt v10.pcl. The thermal cycling protocol consists of a Thermal Mix of 70° C., 40 min; 66° C., 30 sec, Hot Start at 95° C., 2 min, PCR Cycle of 2 cycles of (96° C., 5 s; 64° C., 480 sec), PCR Cycle of 30 cycles of (96° C., 5 s; 62° C., 30 sec), and Melting using a ramp from 60° C. to 95° C. at 1° C./3 s. Data was analyzed using Fluidigm Real-Time PCR Analysis software using the Linear (Derivative) Baseline Correction Method and the Auto (Global) Ct Threshold Method. The C_{q} values determined were exported to Excel for further processing. For each of the patient samples, two independent IFCs were run and the results consolidated by averaging the technical replicates.

To call mutations, Applicants first modelled the background level of expression of the mutated allele by linear regression through assessment of normal B cells known to have absence of the mutation of interest. We then calculated the fraction of the normalized mutant allele over normal plus normalized mutant allele. Cells with this normalized fractional mutant allele below 0.15 were called as ‘normal’, while cells with this normalized fractional mutant allele greater than 0.3 were called as ‘mutant’, and anything in between were called as ‘unclear.’ A threshold of 0.3 was determined by ad-hoc assessment on the negative controls. Applicants restricted subsequent analysis to cells for which we could confidently call ‘normal’ or ‘mutant’ status. Cells for which Applicants did not detect either the mutant or the normal alleles, yielding a normalized mutant allele level of 0/0, were excluded.

### In Vitro Viability Experiments, TRAIL- and Ibrutinib-Induced Apoptosis

After obtaining informed consent, peripheral blood samples were obtained from patients fulfilling diagnostic and immunophenotypic criteria for CLL at MDACC or at DFCI. Consent for samples used in this study was obtained in accordance with the Declaration of Helsinki on protocols that were reviewed and approved by the Institutional Review Boards of MDACC or Dana-Farber/Harvard Cancer Center. Mononuclear cells were isolated from blood samples by utilizing Ficoll-Paque (GE Healthcare, Waukesha, Wis.) density gradient centrifugation according to manufacturer's instructions. Fresh or thawed cryopreserved mononuclear cells were treated with 5 μM ibrutinib (Selleck Chemicals, Houston, Tex.) and/or Super Killer TRAIL (ENZO Biochem, New York, N.Y.) and cell viability was assessed in CD19-positive CLL cells at 24 hour intervals on an LSR Fortessa flow cytometer (BD Biosciences, San Diego, Calif.) after staining with Annexin V-FITC (BD Biosciences), propidium iodine (PI, Sigma, St. Louis, Mo.) and anti-CD19-APC (BD Biosciences). Data analysis was performed on the time point for each sample that exhibited viability closest to 75% in untreated cells.

### Results

### Patients 1-4

### CLL Relapse Following Ibrutinib Therapy

At ibrutinib treatment initiation, Patients 1-4 had advanced stage CLL (Rai stage 3-4, Table 1). Patients 1 and 2 had relapsed diseased after FCR frontline therapy, while Patient 3 and 4 had received multiple lines of prior therapy. CLL samples from all patients harbored high-risk cytogenetic abnormalities (Patients 1 and 3 with del(17p) and Patients 2 and 4 with complex cytogenetic including de/(11q) and del(17p). As the best response to ibrutinib-based therapy, all four experienced partial remissions. Patient 1 demonstrated normalization of hematologic parameters after 183 days, with persistent bone marrow disease (12% residual CLL cells after 448 days on ibrutinib). Patient 2 had normalization of hematologic parameters after 87 days with resolution of lymphadenopathy and splenomegaly, but persistent marrow disease (29% residual CLL cells). Patient 3 achieved a >10-fold reduction but persistently elevated absolute lymphocyte counts (ALC) of approximately 15,000/μL. Patient 4 had normalization of hematologic parameters with resolution of lymphadenopathy and splenomegaly, but persistent lymphocytosis and marrow disease. Progressive disease (PD) on ibrutinib therapy, characterized by increases in lymphocyte counts with a short lymphocyte doubling time (<3 months), along with anemia, thrombocytopenia, and neutropenia, and recurrence of lymphadenopathy and splenomegaly was noted after 983, 176 and 554 days, respectively in Patients 1-3. Patient 4 developed progressive lymphadenopathy, anemia, and thrombocytopenia without worsening lymphocytosis after 669 days of ibrutinib therapy. Patients 1 and 3 proceeded to other forms of therapy, including anti-CD20 mAbs and alternative kinase inhibitors, and were doing well at the time of manuscript preparation (one in remission, one with stable disease), whereas Patient 2 expired from sepsis 63 days after ibrutinib discontinuation, and Patient 4 expired from a hemorrhage 34 days after ibrutinib discontinuation.

TABLE 1
Patient characteristics.

| | Age (yrs)/ | | Pre-ibrutinib | | IGHV | | Best | Time to |
|---|---|---|---|---|---|---|---|---|
| | Gender/ | | FISH | | (M, | | response to | PD on |
| Pt # | Rai stage | Prior therapy | cytogenetics | | U) | Treatment | ibrutinib | ibrutinib |
| 1 | 59/M | FCR | del | (17p), | ND | Ibrutinib | PR | 983 |
| | Rai III | | del | (13q) | | | | |
| 2 | 36/F | FCR, | del | (11q) | U | Ibrutinib + | PR | 176 |
| | Rai IV | R + HDMP | | | | rituximab | | |
| 3 | 85/F | R, BR, CLB, | del | (17p), | U | Ibrutinib | PR | 554 |
| | Rai IV | R + HDMP | del | (13q), | | | | |
| | | | trisomy 12 | | | | | |
| 4 | 58/M | FCR, FR, | del | (17p), | U | Ibrutinib | PR | 669 |
| | Rai IV | CHOP, allo- | del | (11q), | | | | |
| | | Tx, BR, | del | (13q) | | | | |
| | | revlimid, | | | | | | |
| | | ofatumumab | | | | | | |
| 5 | 58/M | FCR, F, R, B | del | (11q), | U | Ibrutinib | PR | 392 |
| | Rai II | | del | (13q) | | | | |

Abbreviations: (gender) M: male; F: female; (prior therapy) FCR: fludarabine, cyclophosphamide, rituximab; BR: bendamustine, rituximab; FR: fludarabine, rituximab; CLB: chlorambucil; R + HDMP: rituximab + high-dose methylprednisolone; F, R, B: single-agent fludarabine, rituximab, bendamustine; allo-Tx: allogeneic stem cell transplantation; CHOP: cyclophosphamide, doxorubicin, vincristine, and prednisone (IGHV) immunoglobulin heavy chain variable region genes, M: mutated, U: unmutated; (best response) PR: partial remission; (time to PD): time to progressive disease.

### Disease Progression is Associated with Marked Clonal Evolution

Whole-exome sequencing and copy number analysis were performed on 2-5 serial peripheral blood CLL samples per patient, from which detection of each somatic mutation and inference of its cancer cell fraction were undertaken, with the exception of Patient 4, from whom only one sample at time of relapse was available.

Patients 1-3 demonstrated distinct patterns of clonal evolution following exposure to ibrutinib. Patient 1's leukemia (FIG. 1A-B) was first studied before starting frontline chemo-immunotherapy with fludarabine, cyclophosphamide, and rituximab (FCR), 3 years prior to start of ibrutinib therapy. The pre-FCR leukemic population was composed predominantly of a clone harboring a mutation in SF3B1 (pG742D), which was eradicated by FCR therapy, and replaced with a clone harboring biallelic inactivation of TP53, trisomy 12, and a new mutation in SF3B1 (p.K666T; CCF of 74%), that drove disease relapse which then instigated ibrutinib initiation. Samples during ibrutinib therapy were collected 1, 2 and 2.7 years after initiating therapy. After two years of continuous ibrutinib treatment, we observed the emergence of 4 PLCG2 mutations (S707F, M1141R, M1141K and D993H) whose expansion involved the entire sample by 2.7 years and was associated with a rapid rise in absolute lymphocyte counts (ALC). All of the detected PLCG2 mutations were novel (8), although a mutation at the S707 site has been previously implicated in ibrutinib resistance (S707Y, ref. 8) and has been shown in vitro to disrupt an auto-inhibitory SH2 domain of PLCG2 (14). We confirmed that these represented 4 distinct subclones by targeted mutation detection in single cells (FIG. 1C, methods).

Applicants obtained estimates for the absolute numbers of cells in each subclone at each time point by integrating CCF with ALC information (methods). A model assuming stable growth rates of the clones throughout the period of ibrutinib therapy fit the ALC counts well, and provided estimates of clonal growth rates during treatment. In comparison to the previously estimated growth rate of CLL cells in a heterogeneous group of patients ranging from −0.29% to 0.71% per day (15), the dominant clone at the start of ibrutinib therapy (clone 4, FIG. 1D) was estimated to decline at a rate of 0.2% (±0.2%) per day, while its progeny clones containing the PLCG2-mutation grew at a rate of 1.5-1.9%±0.1-0.2% per day. By extrapolating the growth rate back to the time of ibrutinib initiation, we estimated that these four clones were already present at the initiation of therapy (clone size ranging from 140 cells to 27,000 cells, FIG. 1E).

The clinical course of Patients 2 and 3 was notable for a shorter interval until disease progression, which suggests different evolutionary dynamics and resistance profiles (16). Indeed, in these patients no mutations in BTK or PLCG2 were observed either by WES or by deep sequencing of the known hotspots (BTK C481 and PLCG2 R665) despite average sequencing depths of 1172X (range 398-2263) and 1126X (range 354-2105), respectively. Instead, in the pre-treatment sample of both these patients, a minor subclone harboring a del(8p) was detected, and in both instances the dominant clone at relapse was a progeny of the del(8p)-positive minor subclone, after the acquisition of additional putative driver mutations in EIF2A (17) and RPS15(18) (Patient 2, FIG. 2A-B), and mutations in known hotspots (18) for the histone acetyltransferase EP300 (Y1397F) and the chromatin regulator MLL2 (Q3892) (Patient 3, FIG. 2D-E). Growth kinetic analysis of Patient 2 showed the del(8p)-containing subpopulation (clone 3) to have a comparable decline rate as the dominant clone at time of ibrutinib initiation (clone 1) (FIG. 2C). However, the progeny of clone 4 which contained the additional mutations in EIF2A and RPS15 (clones 4 and 5), exhibited elevated estimated growth rates of 3.3% and >4.5% per day, respectively, and were estimated to comprise a median of 87,000,000 (or 1 in 1600 cells) cells at treatment initiation. Patient 3 demonstrated a similar picture (FIG. 2F), with the progeny of the del(8p) cells, which contained mutations in EP300 and MLL2, estimated to grow at a rate of ˜4% per day. Together, these findings suggest that the shorter time-to-ibrutinib-failure in Patients 2 and 3 compared with Patient 1 was impacted by both the faster growth kinetics and a larger starting population of resistant cells at treatment initiation.

From Patient 4, only one sample at time of relapse was available, and hence it was not possible to follow changes in the clonal dynamics in this patient. Nonetheless, in this patient, the previously reported BTK-C481S mutation was detected by WES, deep sequencing at the time of relapse, and by RNA-sequencing of the same sample (FIG. 8).

### Detection of Treatment-Resistant Subclones Prior to Ibrutinib Exposure

To experimentally confirm the calculations of clone size at treatment initiation, Applicants developed an ultra-sensitive approach that leverages the ability of droplet-digital amplification technology to evaluate single cells at high throughput. Although bulk quantitative RT-PCR of the mutated allele can detect rare mutated transcripts, it cannot provide information on the actual number of affected cells. Deep targeted sequencing can only affordably detect alleles down to 1 in 100 or 1000 cells, but is prohibitively expensive for detection of rarer events. Droplet technology, on the other hand, can compartmentalize single cells at very high throughputs (>3,000 per second) inside individual “reactors” where enzymatic reactions such as RT-PCR, can be performed on each cell.

To reliably detect rare mutation-bearing cells, we devised a two-stage amplification and quantification approach (FIG. 3A), focusing on transcripts rather than DNA since the likelihood of single-cell drop-out would be less because of greater transcript abundance. The first stage focuses on the sensitive detection of cDNA from cells harboring the specified mutated allele. Single cells are encapsulated in droplets, wherein they undergo lysis and the released mutated transcript can efficiently undergo allele-specific RT-PCR (FIG. 3B-C). For cell populations of 1 in 10³ leukemia cells or greater, we estimated that this first stage of processing would be sufficient for detection of single mutated cells. Indeed, for Patients 2 and 3, we could detect small cell populations bearing mutations associated with the resistant subclone within the pretreatment cells, but not in PBMC from normal adult donors (FIG. 3D). Furthermore, these mutation-bearing cells were expanded in number at the time of relapse (FIG. 9). For Patient 2, we detected the presence of mutated RP515 in 0.06% of pretreatment cells (calculated previously to be 0.06%), while for Patient 3, we detected mutated DGKA in 0.15% cells. Both measurements were at or within a comparable order of magnitude of detection by deep targeted sequencing of these mutations (0.57% for Patient 2/mutated RPS15; 0.07% for Patient 3/mutated DGKA).

For patient 1, the initial detection of cells containing mutant transcripts indicated a frequency of 0.0002%, or 1 in 500,000 (FIG. 3E). As this estimate was based on observation of only seven events, a second stage detection procedure was added for confirmation. In this second stage, pooled mutated amplicons were re-encapsulated using a Poisson distribution to ensure <30% of droplets contain templates. Following digital PCR of the encapsulated templates, the bright droplets were counted by fluorescence detection. For PLCG2-M1141R, we generated a standard curve from PBMC spiked with known numbers of cells from the 30019 cell line, engineered to stably express mutated PLCG2-M1141R, and we could reliably detect 1 in 10⁴, 10⁵ and 10⁶ cells with the PLCG2 mutation, compared to 10⁶ cells without the mutation, or the negative water control. In this fashion, we confirmed detection of 1 in 500,000 pretreatment cells of Patient 1 with mutated PLCG2-M1141R—of similar order of magnitude as our mathematical calculations (CI 1 in 7 million to 1 in 600,000). Altogether, these results confirm that pretreatment samples already contain resistant subclones prior to the initiation of targeted inhibition of BTK, albeit at rare frequencies.

### Patient 5

### Trans-Differentiation From CLL to Histiocytic Sarcoma with Ibrutinib Exposure

Patient 5 also demonstrated clonal evolution but his relapse trajectory was markedly different. At diagnosis, 6 years prior to initiation of ibrutinib, this patient presented with bulky lymphadenopathy and del(11q) and de/(13q) by FISH cytogenetics. He shortly thereafter was treated with frontline FCR, relapsed two years later, and was re-treated with multiple courses of single-agent fludarabine, rituximab, and bendamustine, without any durable responses. Therefore, he proceeded to ibrutinib therapy, and achieved a partial remission, characterized by normalization of the ALC after a transient increase in lymphocytosis and rapid major reduction of his bulky lymph nodes. While still in hematologic remission, he presented at day 392 with a one-week history of fatigue, malaise, muscle and joint aches, night sweats, and low-grade fevers. Evidence for CLL relapse or Richter's transformation was absent since recurrence of bulky lymphadenopathy was not noted on physical examination or CT scans (FIG. 4A), and histopathology examination of bone marrow testing revealed only 2% involvement by CLL cells (compared to 40% before starting ibrutinib therapy). Laboratory studies demonstrated a normal ALC of 1,800/μL. The patient was admitted for treatment of presumed systemic infection and renal failure and received empiric antibiotics and fluid resuscitation, without improvement. Two days after admission, he was transferred to the ICU for multi-organ dysfunction and expired the same day. Autopsy revealed extensive involvement of liver, spleen, lung, kidney and multiple lymph nodes with histiocytic sarcoma (HS). The neoplastic cells were positive for monocyte/macrophage markers CD68 (PGM1) and CD163, but negative for CD1a, CD30, CD5, CD15, CD3, CD45 (LCA), CD19, S-100, and PAX-5 (FIG. 4B). Immunoglobulin heavy chain variable region (IGHV) gene analysis of HS tissue, which tested negative for any B cells by MC, revealed a clonal band (VH3-09), unmutated, characteristic for antigen-experienced B cells, which is the same family and somatic mutation status as originally detected in this patient's CLL cells.

### Genetic Dissection of Clonal Evolution in Patient 5

Three distinct tissues from two time points were evaluated by WES (FIG. 4C). Pre-treatment CLL DNA was extracted from bone marrow, collected before ibrutinib therapy. The progression DNA samples were extracted from lymph node and liver autopsy samples, both of which were confirmed to have involvement by histiocytic sarcoma. As germline comparison, DNA was extracted from uninvolved cardiac muscle. We found that all three samples shared a common set of mutations (e.g., ATM, BRAF and del[11q]), indicative of a common ancestor of the CLL and HS, consistent with the IGHV analysis. A large CLL subclone (CCF of 36%) distinguished by mutations in DMBX1 and DNAJB14 gave rise to the histiocytic sarcoma parent clone which notably contained de/(8p) as well as an NRAS mutation. These mutations define the HS parent as they were shared by HS cells in both the liver and the lymph node samples. Finally, further clonal diversification was observed within the lymph node and the liver samples. For example, all HS cells in the lymph node but not in the liver had the HS parent mutations as well as an EP 300 mutation (N1511S).

### Apoptosis Resistance in CLL Samples with del(8p) in Response to Ibrutinib and/or TRAIL

Having unexpectedly observed del(8p) in the resistance clone of 3 of 5 patients with ibrutinib relapse, we examined the genes in this region more closely. The region of del(8p) in Patients 2, 3 and 5 encompassed TRAIL-R (FIG. 8B), and we observed a decrease in the TRAIL-R mRNA levels corresponding to an increase in the CCF of the del(8p) harboring clone. Previous reports have identified haploinsufficiency of the TRAIL, receptor as a potential target of del(8p)(21). Intriguingly, a potential indirect mechanism that would link TRAIL resistance to positive selection by ibrutinib therapy is suggested by the fact that TRAIL concentrations are higher in circulating blood compared with the lymph node environment (22)(FIG. 8D). Ibrutinib therapy is known to mobilize CLL cells from the lymph node and spleen to the periphery, resulting in lymphocytosis (8). Hence, a potential mechanism by which haplo-insufficiency of the TRAIL-R could provide a survival advantage for CLL cells with this deletion is through the relative insensitivity of CLL cells to cell death in the periphery once they are released from the lymph node and are exposed to higher levels of TRAIL.

To explore this possibility, Applicants quantified TRAIL- and ibrutinib-induced apoptosis in CLL samples with del(8p) and in non-del(8p) controls (del(8p) status verified by FISH (FIGS. 5A-B, FIG. 8). Treatment with ibrutinib (5 μM) resulted in a significant decrease in CLL cell viability in both del(8p) and non-de(8p) controls. In contrast, treatment with TRAIL induced a significant decline in CLL cell viability only in the non-del(8p) samples (FIG. 5C), where a 5% median decrease in cell viability was observed in del(8p) CLL samples (n=6; p=0.14), while non-del(8p) controls (n=9) showed a 16% median decrease in viability (p=0.04). Hence, mono-allelic deletion of chromosome 8p was sufficient to abrogate the positive or negative effects of TRAIL on cell viability in vitro. Combination treatment with ibrutinib and TRAIL further significantly reduced CLL cell viability by a median of 44% (p=0.005, n=9) in non-de/(8p) samples. In contrast, there was only a small, non-significant reduction in median viability by 6% in de/(8p) CLL samples (p=0.09, n=6; see FIG. 5C, lower panel). These data indicate that del(8p) confers partial resistance to apoptosis in response to TRAIL or TRAIL in combination with ibrutinib. These differences were evident in Patient 3 who demonstrated the expected sensitivity to TRAIL in the pre-treatment samples and resistance in the relapse sample, confirming the role of del(8p) in protection from TRAIL-induced apoptosis (FIG. 5D).

### Discussion

The foremost obstacle to effective targeted therapy is the emergence of disease resistance through clonal evolution. BCR-ABL kinase domain mutations in patients with CML, which confer resistance to the tyrosine kinase inhibitor imatinib (23), are well-characterized examples for this mechanism. Reminiscent of the imatinib experience, two reports recently highlighted point mutations in BTK (C481S) (7, 8) that disrupt ibrutinib binding and in its related pathway member PLCG2 (R665W, L845F, S707Y)(8) that can activate the BCR pathway independently from BTK as mechanisms of ibrutinib resistance.

An important question emerging from these data is whether ongoing mutagenesis during ibrutinib therapy led to acquisition of these resistance mutations, or whether this is rather due to expansion of pre-existing sub-clones under therapeutic pressure. Previous studies failed to detect pre-treatment resistance mutations (8). Based on an integrated investigation of the clonal dynamics, growth rate kinetics and experimental detection of rare mutation-bearing cell populations, Applicants analyses provide for the first time, evidence for the presence of substantial diversity of resistant sub-clones at treatment initiation, in line with theoretical predictions (Bozic, I., Nowak, M. A. “Timing and heterogeneity of mutations associated with drug resistance in metastatic cancers” PNAS 2014 111 (45) 15964-15968). In particular, Applicants observed that the genetic composition and kinetics of ibrutinib resistance were dictated by clone size and growth rate of the resistant cells, i.e. the fitness of the resistant clone in relation to other sibling clones (N. Komarova, J A Burger, D Wodarz, Evolution of ibrutinib resistance in chronic lymphocytic leukemia (CLL) PNAS 2014 111 (38) 13906-13911.)

Patient 1 was particularly exemplary: in this patient, Applicants identified 4 distinct PLCG2 mutations, confirmed by RNAseq and deep sequencing validation. Shifts in their relative proportion suggest the presence of 4 distinct sub-clones, with distinct growth rates. The relatively small proportion of these clones at treatment initiation suggests either no fitness advantage or a minor fitness advantage of these mutations in the absence of ibrutinib, which became accentuated by ibrutinib therapy. This patient's leukemia had another instance of convergent evolution with clonal shifts in relation to prior FCR therapy, where a clone containing a SP3B1 mutation was replaced by another clone harboring a different SF3B1 mutation (FIG. 1). This case demonstrates the enormous amount of trial and error that occurs in the process of cancer diversification serving its ability to adapt to therapy.

Notably, while the BTK-C481S mutation and PLCG2 mutations were found in 2 of 5 subjects, the remaining patients revealed a diverse spectrum of mutations present in resistant cells, illustrating the diversity of mutations participating in the disease progression with ibrutinib therapy. The relapse clones of Patients 2, 3 and 5 all arose from parent clones with large deletions of chromosome 8p, previously described to be present in only 5% of the CLL cases and associated with poor prognosis in CLL (26), especially in patients with del (17p)(27). Del(8p) is also a recurrent event in mantle cell lymphoma and other non-Hodgkin lymphomas. As we previously reported, del(8p) likely is a CLL driver that appears later in the evolutionary history of CLL (9, 26). This large region encompasses deletions of the tumor necrosis factor-related apoptosis-inducing ligand (TRAIL) receptor gene loci (21), which we confirmed to he downregulated with RNAseq expression data from Patients 2 and 3 (Supplementary Tab. S3). Monoallelic deletion of TRAM-R1/2 (TNFRSF10A/B, also called death receptor 4/5 [DR4/DR5]) can antagonize TRAIL-induced apoptosis in B-NHL (21), suggesting that TRAIL-R1/2 may function as tumor suppressors. Our functional data support TRAIL receptor haplo-insufficiency as a potential resistance mechanism. We noted robust TRAIL-induced apoptosis in CLL samples with intact 8p, but reduced or absent TRAIL-induced apoptosis in samples with del(8p), demonstrating that mono-allelic deletion was sufficient to abrogate the pro-apoptotic effects of TRAIL. This was confirmed in serial CLL cell samples from Patient 3, where preserved sensitivity to TRAIL was noted in a pre-treatment sample, and resistance to TRAIL in the relapse sample (FIG. 5). Interestingly, the analysis of clonal kinetics revealed that the del(8p) clones also declined with ibrutinib (albeit in a slower rate), and are replaced by their progeny, containing additional somatic alterations. This raises the intriguing hypothesis that, while del(8p) provides a fitness advantage in the absence of ibrutinib, it needs to co-operate with additional lesions to achieve the resistant phenotype.

Histiocytic sarcomas are myeloid tumors, which rarely evolve in patients with NHL and CLL as a result of cross-lineage trans-differentiation (28). As in the case of Patient 5, these exceedingly rare cases of histiocytic sarcomas are clonally related to the B cell malignancy, based on shared IGHV immunoglobulin gene rearrangements and additional shared mutations. These cases have been interpreted as signs of lineage plasticity of the underlying B-cell neoplasm, a phenomenon that was originally recognized in mouse models (29). In these models, enforced expression of the transcription factors C/EBPα and C/EBPβ promoted transdifferentiation of B-cells into macrophages. In humans, transdifferentiation of lymphoid malignancies was found to be association with mutations of NRAS and BRAF. Chen et al. described a case of Langerhans cell sarcoma (LCS), transdifferentiated from CLL that carried a BRAF V600E mutation (30). Buser et al. reported about transdifferentiation of a T lymphoblastic lymphoma into an indeterminate dendritic cell tumor carrying a G13D mutation of the NRAS gene (31). Interestingly, our patient had both, BRAF and NRAS mutations that may be involved in the transdifferentiation process.

The fact that the CLL clone at the time of trans-differentiation remained in remission is characteristic, as is the poor prognosis, with survival generally of only days to weeks. This case of histiocytic sarcoma trans-differentiation, with tumor cells no longer dependent on BCR signaling, indicates that ibruitinib resistance may be a more complex process than initially thought, and this potent therapy may serve as a strong evolutionary drive to differentiate away from the B cell identity and its accompanying dependency on BCR signaling.

Based on these findings Applicants have developed a novel analytic framework that is widely applicable across cancer and disease—detections of subclonal populations before treatment and further applying frequent serial clonal analysis can inform practitioners regarding the clone-specific decline/growth kinetics as they occur in patients. This type of analysis provides vital information regarding the fitness of different genetic lesions with and without therapy, which may be immensely beneficial to the design of the next generation of therapeutic approaches to overcome the evolutionary capacity of cancer and disease.

### REFERENCES

1. Burger J A, Chiorazzi N. B cell receptor signaling in chronic lymphocytic leukemia. Trends Immunol. 2013; 34:592-601.

2. Friedberg J W, Sharman J, Sweetenham J, Johnston P B, Vose J M, Lacasce A, et al. Inhibition of Syk with fostamatinib disodium has significant clinical activity in non-Hodgkin lymphoma and chronic lymphocytic leukemia. Blood. 2010; 115:2578-85.

3. Byrd J C, Furman R R, Coutre S E, Flinn I W, Burger J A, Blum K A, et al. Targeting BTK with ibrutinib in relapsed chronic lymphocytic leukemia. The New England journal of medicine. 2013; 369:32-42.

4. Furman R R, Sharman J P, Coutre S E, Cheson B D, Pagel J M, Hillmen P, et al. Idelalisib and rituximab in relapsed chronic lymphocytic leukemia. The New England journal of medicine. 2014; 370:997-1007.

5. Honigberg L A, Smith A M, Sirisawad M, Verner E, Loury D, Chang B, et al. The Bruton tyrosine kinase inhibitor PCI-32765 blocks B-cell activation and is efficacious in models of autoimmune disease and B-cell malignancy. Proceedings of the National Academy of Sciences of the United States of America. 2010; 107:13075-80.

6. Woyach J A, Furman R R, Liu T M, Ozer H G, Zapatka M, Ruppert A S, et al. Resistance Mechanisms for the Bruton's Tyrosine Kinase Inhibitor Ibrutinib. The New England journal of medicine. 2014.

7. Furman R R, Cheng S, Lu P, Setty M, Perez A R, Guo A, et al. Ibrutinib Resistance in Chronic Lymphocytic Leukemia. The New England journal of medicine. 2014.

8. Woyach J A, Furman R R, Liu T M, Ozer H G, Zapatka M, Ruppert A S, et al. Resistance mechanisms for the Bruton's tyrosine kinase inhibitor ibrutinib. The New England journal of medicine. 2014; 370:2286-94.

9. Landau D A, Carter S L, Stojanov P, McKenna A, Stevenson K, Lawrence M S, et al. Evolution and impact of subclonal mutations in chronic lymphocytic leukemia. Cell. 2013; 152:714-26.

10. Cibulskis K, Lawrence M S, Carter S L, Sivachenko A, Jaffe D, Sougnez C, et al. Sensitive detection of somatic point mutations in impure and heterogeneous cancer samples. Nat Biotechnol. 2013; 31:213-9.

11. Robinson J T, Thorvaldsdottir H, Winckler W, Guttman M, Lander E S, Getz G, et al. Integrative genomics viewer. Nat Biotechnol. 2011; 29:24-6.

12. Carter S, Meyerson M, Getz G. Accurate estimation of homologue-specific DNA concentration-ratios in cancer samples allows long-range haplotyping. Nature Precedings. 2011: hdl.handle.net/10101/npre2011.6494.1.

13. Carter S L, Cibulskis K, Helman E, McKenna A, Shen H, Zack T, et al. Absolute quantification of somatic DNA alterations in human cancer. Nat Biotechnol. 2012; 30:413-21.

14. Zhou Q, Lee G S, Brady J, Datta S, Katan M, Sheikh A, et al. A hypermorphic missense mutation in PLCG2, encoding phospholipase Cgamma2, causes a dominantly inherited autoinflammatory disease with immunodeficiency. Am J Hum Genet. 2012; 91:713-20.

15. Messmer B T, Messmer D, Allen S L, Kolitz J E, Kudalkar P, Cesar D, et al. In vivo measurements document the dynamic cellular kinetics of chronic lymphocytic leukemia B cells. The Journal of clinical investigation. 2005; 115:755-64.

16. Chiron D, Di Liberto M, Martin P, Huang X, Sharman J, Blecua P, et al. Cell-cycle reprogramming for PI3K inhibition overrides a relapse-specific C4815 BTK mutation revealed by longitudinal functional genomics in mantle cell lymphoma. Cancer Discov. 2014; 4:1022-35.

17. Fritsch R M, Schneider G, Saur D, Scheibel M, Schmid R M. Translational repression of MCL-1 couples stress-induced eIF2 alpha phosphorylation to mitochondrial apoptosis initiation. The Journal of biological chemistry. 2007; 282:22551-62.

18. Lawrence M S, Stojanov P, Mermel C H, Robinson J T, Garraway L A, Golub T R, et al. Discovery and saturation analysis of cancer genes across 21 tumour types. Nature. 2014; 505:495-501.

19. Guo M T, Rotem A, Heyman J A, Weitz D A. Droplet microfluidics for high-throughput biological assays. Lab Chip. 2012; 12:2146-55.

20. Mazutis L, Gilbert J, Ung W L, Weitz D A, Griffiths A D, Heyman J A. Single-cell analysis and sorting using droplet-based microfluidics. Nat Protoc. 2013; 8:870-91.

21. Rubio-Moscardo F, Blesa D, Mestre C, Siebert R, Balasas T, Benito A, et al. Characterization of 8p21.3 chromosomal deletions in B-cell lymphoma: TRAIL-R1 and TRAIL-R2 as candidate dosage-dependent tumor suppressor genes. Blood. 2005; 106:3214-22.

22. Herbeuval J P, Nilsson J, Boasso A, Hardy A W, Vaccari M, Cecchinato V, et al. HAART reduces death ligand but not death receptors in lymphoid tissue of HIV-infected patients and simian immunodeficiency virus-infected macaques. AIDS. 2009; 23:35-40.

23. Shah N P, Nicoll J M, Nagar B, Gorre M E, Paquette R L, Kuriyan J, et al. Multiple BCR-ABL kinase domain mutations confer polyclonal resistance to the tyrosine kinase inhibitor imatinib (STI571) in chronic phase and blast crisis chronic myeloid leukemia. Cancer Cell. 2002; 2:117-25.

24. Bozic I, Allen B, Nowak M A, Dynamics of targeted cancer therapy. Trends Mol Med. 2012; 18:311-6.

25. Bozic I, Reiter J G, Allen B, Antal T, Chatterjee K, Shah P, et al. Evolutionary dynamics of cancer in response to targeted combination therapy. Elife. 2013; 2:e00747.

26. Brown J R, Hanna M, Tesar B, Werner L, Pochet N, Asara T M, et al. Integrative genomic analysis implicates gain of PIK3CA at 3g26 and MYC at 8q24 in chronic lymphocytic leukemia. Clin Cancer Res. 2012; 18:3791-802.

27. Forconi F, Rinaldi A, Kwee I, Sozzi E, Raspadori D, Rancoita P M, et al. Genome-wide DNA analysis identifies recurrent imbalances predicting outcome in chronic lymphocytic leukaemia with 17p deletion. British journal of haematology. 2008; 143:532-6.

28. Shao H, Xi L, Raffeld M, Feldman A L, Ketterling R P, Knudson R, et al. Clonally related histiocytic/dendritic cell sarcoma and chronic lymphocytic leukemia/small lymphocytic lymphoma: a study of seven cases. Mod Pathol. 2011; 24:1421-32.

29. Xie H, Ye M, Feng R, Graf T. Stepwise reprogramming of B cells into macrophages. Cell. 2004; 117:663-76.

30. Chen W, Jaffe R, Zhang L, Hill C, Block A M, Sait S, et al. Langerhans Cell Sarcoma Arising from Chronic Lymphocytic Lymphoma/Small Lymphocytic Leukemia: Lineage Analysis and BRAF V600E Mutation Study. N Am J Sci. 2013; 5:386-91.

31. Buser L, Bihl M, Rufle A, Mickys U, Tavoriene I, Griskevicius L, et al. Unique composite hematolymphoid tumor consisting of a pro-T lymphoblastic lymphoma and an indeterminate dendritic cell tumor: evidence for divergent common progenitor cell differentiation. Pathobiology. 2014; 81:199-205.

32. Secchiero P, Tiribelli M, Barbarotto E, Celeghini C, Michelutti A, Masolini P, et al. Aberrant expression of TRAIL in B chronic lymphocytic leukemia (B-CLL) cells. J Cell Physiol. 2005; 205:246-52.

33. Oh H, Siano B, Diamond S. Neutrophil isolation protocol. J Vis Exp. 2008.

34. Wierda W G, O'Brien S, Wang X, Faderl S, Ferrajoli A, Do K A, et al. Multivariable model for time to first treatment in patients with chronic lymphocytic leukemia. J Clin Oncol. 2011; 29:4088-95.

35. Dohner H, Stilgenbauer S, Benner A, Leupolt E, Krober A, Bullinger L, et al. Genomic aberrations and survival in chronic lymphocytic leukemia. The New England journal of medicine. 2000; 343:1910-6.

36. Lin K I, Tam C S, Keating M J, Wierda W G, O'Brien S, Lerner S, et al. Relevance of the immunoglobulin VH somatic mutation status in patients with chronic lymphocytic leukemia treated with fludarabine, cyclophosphamide, and rituximab (FCR) or related chemoimmunotherapy regimens. Blood. 2009; 113:3168-71.

37. Fais F, Ghiotto F, Hashimoto S, Sellars B, Valetto A, Allen S L, et al. Chronic lymphocytic leukemia B cells express restricted sets of mutated and unmutated antigen receptors. The Journal of clinical investigation. 1998; 102:1515-25.

38. Fisher S, Barry A, Abreu J, Minie B, Nolan J, Delorey T M, et al. A scalable, fully automated process for construction of sequence-ready human exome targeted capture libraries. Genome Biol. 2011; 12:R1.

39. Li H, Durbin R. Fast and accurate long-read alignment with Burrows-Wheeler transform. Bioinformatics. 2010; 26:589-95.

40. Cibulskis K, Lawrence M S, Carter S L, Sivachenko A, Jaffe D, Sougnez C, et al. Sensitive detection of somatic point mutations in impure and heterogeneous cancer samples. Nat Biotechnol. 2013; 31:213-9.

41. Robinson J T, Thorvaldsdottir H, Winckler W, Guttman M, Lander E S, Getz G, et al. Integrative genomics viewer. Nat Biotechnol. 2011; 29:24-6.

42. Carter S, Meyerson M, Getz G, Accurate estimation of homologue-specific DNA concentration-ratios in cancer samples allows long-range haplotyping. Nature Precedings. 2011:hdl.handle.net/10101/npre.2011.6494.1.

43. Lohr J G, Stojanov P, Lawrence M S, Auclair D, Chapuy B, Sougnez C, et al. Discovery and prioritization of somatic mutations in diffuse large B-cell lymphoma (DLBCL) by whole-exome sequencing. Proceedings of the National Academy of Sciences of the United States of America. 2012; 109:3879-84.

44. Carter S L, Cibulskis K, Helman E, McKenna A, Shen H, Zack T, et al. Absolute quantification of somatic DNA alterations in human cancer. Nat Biotechnol. 2012; 30:413-21.

45. Landau D A, Carter S L, Stojanov P, McKenna A, Stevenson K, Lawrence M S, et al. Evolution and impact of subclonal mutations in chronic lymphocytic leukemia. Cell. 2013; 152:714-26.

46. McFadden D G, Papagiannakopoulos T, Taylor-Weiner A, Stewart C, Carter S L, Cibulskis K, et al. Genetic and clonal dissection of murine small cell lung carcinoma progression by genome sequencing. Cell. 2014; 156:1298-311.

47. Herman S E, Niemann C U, Farooqui M, Jones J, Mustafa R Z, Lipsky A, et al. ibrutinib-induced lymphocytosis in patients with chronic lymphocytic leukemia: correlative analyses from a phase II study. Leukemia. 2014.

48. Qin D, Xia Y, Whitesides G M. Soft lithography for micro- and nanoscale patterning. Nat Protoc. 2010; 5:491-502.

49. Link D R, Grasland-Mongrain E, Duri A, Sarrazin F, Cheng Z, Cristobal G, et al. Electric control of droplets in microfluidic devices. Angew Chem Int Ed Engl. 2006; 45:2556-60.

50. Livak K J, Wills Q F, Tipping A J, Datta K, Mittal R, Goldson A J, et al. Methods for qPCR gene expression profiling applied to 1440 lymphoblastoid single cells. Methods. 2013; 59:71-9.

51. Marius S, Vargas-Gold D, Tyagi S, Kramer F R, inventors; Highly selective nucleic acid amplification primers 2014.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A method of individualized or personalized treatment for a disease undergoing clonal evolution and for preventing relapse after treatment in a patient in need thereof comprising:
(a) determining mutations present in a disease cell fraction from the patient before administration of a therapy;
(b) determining subclonal populations within the disease cell fraction;
(c) selecting at least one subclonal population to treat; and
(d) treating the patient with a therapy comprising administering at least one component, wherein each selected subclonal population does not contain a mutation associated with resistance to the at least one component of the therapy.

2. The method of claim 1, further comprising determining mutations and subclonal populations on at least one time point after administration of the therapy.

3. The method of claim 2, wherein the treatment is adjusted if new mutations associated with resistance are detected in a subclonal population.

4. The method of claim 2, wherein selecting at least one subclonal population to treat comprises determining subclone-specific decline and/or growth kinetics, wherein the treatment is adjusted if there is an increase in at least one subclone.

5. The method according to claim 1, wherein the therapy comprises administering at least two components, wherein each selected subclonal population is targeted by at least one component of the therapy and wherein each selected subclonal population does not contain a mutation associated with resistance to at least one component of the therapy.

6. The method according to claim 1, wherein selecting at least one subclonal population to treat comprises determining the copy number of each subclonal population.

7. The method according to claim 1, wherein the mutations are somatic mutations.

8. The method according to claim 1, wherein the disease is cancer.

9. The method of claim 8, wherein the cancer is chronic lymphocytic leukemia (CLL); wherein the presence of a mutation in a subclonal population of one or more genes selected from the group consisting of ATM, BRAF, DMBX1, del(8p), del(11q), del(13q), DNAJB14, EJF2A, EP300, MLL2, NRAS, RPS15,and SF3B1 indicates the person is insensitive to a Bruton's tyrosine kinase (BTK) inhibitor; and wherein the therapy comprises administering at least one component other than a Bruton's tyrosine kinase (BTK) inhibitor in addition to or independent of a Bruton's tyrosine kinase (BTK) inhibitor, if there is a mutation in a subclonal population of one or more genes selected from the group consisting of ATM, BRAF, DMBX1, del(8p), del(11q), del(13q), DNAJB14, EIF2A, EP300, MLL2, NRAS, RPS15, and SF3B1.

10. The method according to claim 1, wherein step (a) comprises:
(i) obtaining a blood, bone marrow, saliva or tissue sample from the patient; (ii) isolating DNA from the blood, bone marrow, saliva or tissue sample; and (iii) genotyping the DNA; or
droplet-based detection of single cells by RT-PCR; or
whole-exome sequencing (WES) and/or genome-wide copy number profiling; or
identifying mutations with an algorithm or
allele-specific analysis; or
deep sequencing and targeted re-sequencing with microfluidic PCR.

11. (canceled)

12. The method of claim 10, wherein the tissue sample is a formalin-fixed, paraffin-embedded (FFPE) tissue section.

13-16. (canceled)

17. The method of claim 10, wherein the step (a) is allele-specific analysis, wherein the allelic fractions are converted into disease cell fractions.

18. The method of claim 17, wherein step (b) comprises:
clustering the disease cell fractions to delineate distinct subclonal populations that contain multiple subclonal mutations and to infer the phylogenetic relationships between these populations; or
immunohistochemical (IHC) staining, fluorescent in situ hybridization (FISH) chromosome analysis, and/or immunoglobulin hypervariable (IGHV) gene region mutation analysis.

19. (canceled)

20. The method of claim 9,
wherein
the mutation in SF3B1 is pG742D; or
the mutation in SF3B1 is p.K666T; or
the mutation is a del(8p) mutation; or
the mutation is a driver mutation in EIF2A and/or RPS15; or
the mutation in EP300 is Y1397F; or
the mutation in MLL2 is Q3892; or
the mutation is in EIF2A and/or RPS15; or
the mutation is in EP300 and/or MLL2; or
the mutation is a del(11q) and/or del(13q) mutation; or
the mutation is a ATM, BRAF and/or del[11q] mutation; or
the mutation in EP300 is N1511S.

21. The method of claim 1, wherein mutations comprise a mutation in TP53, and wherein the mutation is biallelic inactivation of TP53; or
wherein mutations comprise a mutation in PLCG2, and wherein the mutation is S707F, M1141R, M1141K and/or D993H.

22-32. (canceled)

33. The method of claim 1, wherein the therapy is chemotherapy, a monoclonal antibody, a targeted therapy, a stem cell transplant, leukapheresis, surgery, radiation therapy or a combination thereof.

34. The method of claim 33, wherein the chemotherapy is a purine analog, an alkylating agent, a corticosteroid or other chemotherapy drug.

35. The method of claim 34, wherein the purine analog is bine (Fludara®), pentostatin (Nipent®), or cladribine (2-CdA, Leustatin®); or wherein the alkylating agent is chlorambucil (Leukeran®), cyclophosphamide (Cytoxan®), or bendamustine (Treanda®); or wherein the corticosteroid is prednisone, methylprednisolone, or dexamethasone; or wherein the other chemotherapy drug is doxorubicin (Adriamycin®), methotrexate, oxaliplatin, vincristine (Oncovin®), etoposide (VP-16), or cytarabine (ara-C).

36-38. (canceled)

39. The method of claim 33, wherein the monoclonal antibody targets a CD20 antigen or a CD52 antigen; or wherein the targeted therapy is Idelalisib (Zydelig®).

40. The method of claim 39, wherein the monoclonal antibody targets a CD20 antigen or a CD52 antigen, and wherein the monoclonal antibody is Rituximab (Rituxan), Obinutuzumab (Gazyva™), Ofatumumab (Arzerra®), or Alemtuzumab (Campath®).

41. (canceled)
